(19) United States
(12) Patent Application Publication
(10) Pub. No.: US 2025/0177478 A1
(43) Pub. Date: Jun. 5, 2025

(54) G ALPHA Q/11 INHIBITORS FOR USE AS AN ANALGESIC

(71) Applicant: University of Florida Research Foundation, Incorporated, Gainesville, FL (US)

(72) Inventor: Kirill Martemyanov, Gainesville, FL (US)

(21) Appl. No.: 18/843,813

(22) PCT Filed: Mar. 28, 2023
(86) PCT No.: PCT/US2023/016509

§ 371 Date: Sep. 4, 2024

Related U.S. Application Data
(60) Provisional application No. 63/324,879, filed on Mar. 29, 2022.

(51) Int. Cl. A61K 38/15; A61K 9/00; A61K 31/4985; A61K 45/06; A61P 25/04; C07K 16/18; C12N 9/22; C12N 15/11; C12N 15/113

(52) U.S. Cl. CPC A61K 38/15; A61K 9/0085; A61K 31/4985; A61K 45/06; A61P 25/04; C07K 16/18; C12N 9/22; C12N 15/111; C12N 15/113; C12N 2310/11; C12N 2310/14; C12N 2310/20

(57) ABSTRACT

Described are compositions and methods for treating pain and/or increasing opioid therapy efficacy and ameliorating opioid use disorder. The compositions and methods target Gα_{g/11} signaling.

### CROSS-REFERENCE TO RELATED APPLICATIONS

This application claims priority to U.S. provisional application Ser. No. 63/324,879, filed Mar. 29, 2022, which is incorporated herein by reference.

### STATEMENT REGARDING FEDERALLY SPONSORED RESEARCH OR DEVELOPMENT

This invention was made with government support under grant number DA036596, awarded by the National Institutes of Health. The government has certain rights in the invention.

### BACKGROUND

Opioids can be effective analgesics, but have unwanted side-effects including addiction, dependence, and respiratory depression. Opioids activate μ-opioid receptors (MOR) to produce analgesia. Propagation of MOR signals may be regulated by other signaling systems.

The marked increase in misuse of prescription opioids has greatly affected our society. One potential solution is to develop analgesics that act at targets other than opioid receptors. These can then be either used as a stand-alone therapeutic or to modify the action of opioid drugs improving their safety profile. Previous research showed that activation of Gg/11 proteins by G protein-coupled receptors has pro-nociceptive properties.

G protein coupled receptors (GPCRs) constitute the largest family of cell surface receptors with immense roles in nearly all known physiological processes including nociception, cardiovascular function and inflammation among many others. Accordingly, GPCRs are frequently targeted by small molecule drugs for therapeutic benefits. However, the pleiotropic effects associated with their activation or inhibition also often lead to unwanted side effects. Many GPCR actions are mediated by heterotrimeric G proteins. GPCRs activate G proteins by catalyzing their nucleotide exchange leading to the release of Gβγ subunits from Gα. Both active Gα-GTP and free Gβγ transduce signal by interacting with a range of downstream effector molecules to elicit a cellular response. There are 12 Gα subunits in mammals, grouped into 5 subfamilies (Gα_{5/olf}, Gα_{i/o}, Gα_{q/11}, Gα_{12/13} and Gα₁₅) and characterized by unique properties and selectivity with which they regulate their effectors. Studies with knockout mice indicate that individual G protein channels selectively contribute to various aspects of GPCR signaling and physiological reactions controlled by them.

Both synergistic and opposing influences of different G proteins in regulating their effector molecules has been observed. Pharmacological targeting of individual G proteins is emerging as an attractive strategy with therapeutic potential (Campbell A P et al. “Targeting G protein-coupled receptor signaling by blocking G proteins.” Nat Rev Drug Discov (2018) 17:789-803).

One particularly prominent pharmacological area of need is the modulation of pain. GPCR signaling is involved in this process with many receptors affecting both ascending and descending nociceptive circuits and regulating various pain states. The mu-opioid receptor (MOR) is targeted by opioid drugs to produce strong analgesia. Notably, several other GPCR systems also produce powerful analgesia (Stone L S et al. “In search of analgesia: emerging roles of GPCRs in pain.” Mol Interv (2009) 9:234-251). However, most of these also trigger unwanted side effects including, dependence, somatic, dysphoria prompting the search for alternative analgesic strategies.

In contrast to largely anti-nociceptive effects associated with activation of Gi/o receptors, triggering Gq and Gs signaling generally leads to opposite outcomes, i.e. hyperalgesia, sensitization to pain, allodynia. Interestingly, increased expression of Gq and G11 proteins has been reported in animal models of pain (Belmadani A et al. “Activation of Keratinocyte Gq-linked G-Protein Coupled Receptors Regulates Degeneration of Cutaneous Nerves.” The Journal of Pain (2021) 22:581 and Saika F et al. “Chemogenetic Activation of CX3CR1-Expressing Spinal Microglia Using Gq-DREADD Elicits Mechanical Allodynia in Male Mice. Cells (2021) 10). Genetic studies in mice indicate that loss of G_{q} and G₁₁ results in reduced pain hypersensitivity in chronic pain states (Tappe-Theodor A et al. “Gα(_{q/11}) signaling tonically modulates nociceptor function and contributes to activity-dependent sensitization.” Pain (2012) 153:184-196). Moreover, knockout of Gα_{q} modulates properties of nociceptors, reduces basal pain sensitivity as well as pain sensitizing effects associated with activation of several Gα_{q/11}-coupled GPCRs (Wirotanseng L N et al. “Gq rather than G11 preferentially mediates nociceptor sensitization.” Molecular pain (2013) 9:54-54).

### SUMMARY

Described are Gα_{q/11} antagonists, pharmaceutical compositions containing Gα_{q/11} antagonists, and methods of using the Gα_{q/11} antagonists and pharmaceutical compositions to treat pain or to mediate an analgesic effect mediated by a μ-opioid receptor (MOR). The Gα_{q/11} inhibitor can be, but is not limited to, YM-254890, FR-900359 (UBO-QIC), GQ262, or GQ127, an nucleic acid-based inhibitor, or an anti-Gα_{q/11} antibody or a Gα_{q/11}-binding fragment thereof. A nucleic acid-based inhibitor can comprise a RNA interference (RNAi) polynucleotide or an antisense oligonucleotide (ASO) that targets the GNA [symbol] gene or the GNA11 gene. An RNAi polynucleotide can be, but is not limited to, an siRNA, an shRNA or a nucleic acid encoding an siRNA or shRNA. Contacting a cell with, or introducing into the cell, one or more RNAi polynucleotides or one or more antisense oligonucleotides can be used to inhibit expression of the GNA [symbol] gene (encoding Gα_{q}) or GNA11 gene (encoding Gα₁₁) in the cell. Inhibiting expression of the GNA [symbol] and/or GNA₁₁ leads to decreased Gα_{q/11} activity in the cell. In some embodiments, a nucleic acid-based inhibitor comprises a CRISPR-based system. In some embodiments, a pharmaceutical composition further contain an opioid. The opioid drug can be, but is not limited to, oxycodone, hydrocodone, morphine, codeine, dihydrocodeine, fentanyl, pethidine, hydromorphone, buprenorphine, or methadone. Gα_{q/11} inhibition can be used to suppresses the activity of nociceptor neurons. Alone or in combination with an opioid, Gα_{q/11} inhibition blocks transmission of noxious stimuli. Pharmacological blockage of Gα_{q/11} can be used for pain management and/or enhancement of opioid potency and safety. The subject can be, but is not limited to, a human subject.

Described is the use of a Gα_{q/11} inhibitor for the treatment of pain, to reduce nociception, and/or to enhance the safety and/or efficacy of an opioid analgesic. The Gα_{q/11} inhibitor can be, but is not limited to, YM-254890, FR-900359 (UBO-QIC), GQ262, or GQ127, an nucleic acid-based inhibitor, or an anti-Gα_{q/11} antibody or a Gog/11-binding fragment thereof. A nucleic acid-based inhibitor can comprise a RNA interference (RNAi) polynucleotide or an antisense oligonucleotide (ASO) that targets the GNA [symbol] or the GNA11 gene. An RNAi polynucleotide can be, but is not limited to, an siRNA, an shRNA or a nucleic acid encoding an siRNA or shRNA. Contacting a cell with, or introducing into the cell, one or more RNAi polynucleotides or one or more antisenses oligonucleotide inhibits expression of the GNA [symbol] gene and/or the GNA11 gene in the cell. Inhibiting expression of the GNA [symbol] and/or GNA11 genes leads to decreased Gog/11 activity in the cell. In some embodiments, a nucleic acid-based inhibitor comprises a CRISPR-based system. The opioid analgesic (opioid drug) can be, but is not limited to, oxycodone, hydrocodone, morphine, codeine, dihydrocodeine, fentanyl, pethidine, hydromorphone, buprenorphine, or methadone. The Gα_{q/11} inhibitor can be administered to the subject prior to an initial dose of the opioid drug, at the same time as an initial dose of the opioid drug, at the same time as one or more subsequent doses of the opioid drug, or after an initial dose of the opioid drug. Increasing efficacy of an opioid therapy can comprise: enhancing maximal response to the opioid, increasing response to a given dose of opioid, enhancing the analgesic effect of low dose of the opiate, decreasing the amount of the opioid required to provide an effective analgesic response, increasing duration of analgesic response to the opioid, and/or decreasing tolerance of the opioid. Increasing efficacy of an opioid therapy can also comprise: increasing safety of an opioid drug, and/or decreasing risk of addiction or dependence on the opioid drug. The subject can be, but is not limited to, a human subject.

Described are methods of suppressing excitability of DRG nociceptors in a subject comprising, administering a Gα_{q/11} inhibitor or a Gα_{q/11} inhibitor and an opioid analgesic to the subject. The Gα_{q/11} inhibitor can be, but is not limited to, YM-254890, FR-900359 (UBO-QIC), GQ262, or GQ127, an nucleic acid-based inhibitor, or an anti-Gag antibody or a Gα_{q}-binding fragment thereof and/or an anti-Gα₁₁ antibody or a Gα₁₁-binding fragment thereof. The opioid analgesic (opioid drug) can be, but is not limited to, oxycodone, hydrocodone, morphine, codeine, dihydrocodeine, fentanyl, pethidine, hydromorphone, buprenorphine, or methadone. The Gα_{g/11} inhibitor can be administered to the subject prior to an initial dose of the opioid drug, at the same time as an initial dose of the opioid drug, at the same time as one or more subsequent doses of the opioid drug, or after an initial dose of the opioid drug. The subject can be, but is not limited to, a human subject

The Gα_{q/11} inhibitor can be administered to a subject by systemic administration, intravenous administration, subcutaneous administration, local administration, epidural administration, or intrathecal administration. In some embodiments, the Gα_{q/11} inhibitor is administered by epidural administration or intrathecal administration. In some embodiments, the Gα_{q/11} inhibitor is YM-254890 and the YM-254890 is administered by epidural administration or intrathecal administration.

Described is the use of local inhibition of Gα_{q/11} signaling in the spinal cord to provide analgesic effect. In some embodiments, Gα_{q/11} blockade is combined with an opioid. The opioid can be administered by any means typically used to administer opioid, including, but not limited to, systemic administration. Combining Gα_{q/11} blockade with an opioid drug enhances efficacy of opioid administration in treating pain. The opioid drug can be, but is not limited to, oxycodone, hydrocodone, morphine, codeine, dihydrocodeine, fentanyl, pethidine, hydromorphone, buprenorphine, or methadone.

Described are methods for inhibiting Gα_{q/11} signaling. The methods comprise administering to a subject a therapeutically effective amount of any one or more of Gα_{q/11} inhibitors. Administering a therapeutically effective amount of a Gα_{q/11} inhibitor includes, but is not limited to, administering to the subject a pharmaceutical composition that contains the therapeutically effective amount of the Gα_{q/11} inhibitor. A Gα_{q/11} inhibitor can be used to enhance an analgesic effect mediated by the μ-opioid receptor (MOR) in a subject. Inhibiting Gα_{q/11} signaling can lead to stimulation or enhancement of MOR mediated analgesic effect in the subject. Inhibiting Gα_{q/11} signaling can be used to reduce opioid tolerance. Inhibiting Gα_{q/11} signaling is also expected to ameliorate withdrawal and thus be beneficial for combating dependence by facilitating opioid abstinence.

Systemic administration of a Gα_{q/11} inhibitor can be used to produce antinociceptive effects an/or to augment opioid drug analgesia. Local administration Gα_{q/11} inhibitor can also be used to produce antinociceptive effects an/or to augment opioid drug analgesia. Local administration can be, but is not limited to, epidural injection and intrathecal injection. In some embodiments, local administration provides for lasting analgesia and/or augments the extent and/or duration of anti-nociceptive effects of opioid drug without any significant effects on locomotor behavior.

In some embodiments, a subject is administered an opioid drug for pain relief and a Gα_{q/11} inhibitor, wherein the Gα_{q/11} inhibitor enhances the effect of the opioid drug. The opioid drug can be administered to the subject prior to, simultaneously with, or subsequent to administration of the one or more Gα_{q/11} inhibitor. In some embodiments, the opioid drug and the Gα_{q/11} inhibitor are formulated together. In some embodiments, the opioid drug and the Gα_{q/11} inhibitor antagonists are formulated for separate administration. The opioid drug can be, but is not limited to, oxycodone, hydrocodone, morphine, codeine, dihydrocodeine, fentanyl, pethidine, hydromorphone, buprenorphine, or methadone. The subject can be, but is not limited to, a human.

A further understanding of the nature and advantages of the present invention may be realized by reference to the remaining portions of the specification and claims.

### BRIEF DESCRIPTION OF THE DRAWINGS

FIG. 1. The effect of systemic subcutaneous administration of YM-254890 (YM) on nociception and locomotion. (A) Dose-response effect of different concentrations of subcutaneous YM (0.1, 0.3, 0.5 and 1 mg/kg) and vehicle was tested on the hotplate test after 30 min of administration. Unpaired two-tailed Student's t test of YM (0.5 mg/kg), p<0.05, and YM (1.0 mg/kg), p<0.05. (B) Effect of combined administration of subcutaneous YM (0.25 and 0.5 mg/kg) with a single dose of subcutaneous morphine (5 mg/kg) on the hotplate test after 30 min of morphine administration. YM was administered 10 min before the administration of morphine. Treatment: F_{(1, 10)}=59.06; dose: F_{(1, 10)}=13.21; and interaction: F_{(1, 10)}=12.08. Two-way analysis of variance (ANOVA) with Bonferroni's post hoc test. (C) Changes in cumulative locomotor activity during 120 min of observation in open-field test by subcutaneous administration of YM (0.5 mg/kg), morphine (5 mg/kg) and YM (0.5 mg/kg, 10 min before morphine) with morphine (5 mg/kg) and vehicle-treated mice. Treatment: F_{(1, 19)}=26.02; effect of morphine: F_{(1, 19)}=3.71; and interaction: F_{(1, 19)}=6.2. Two-way ANOVA with Bonferroni's post hoc test. In all panels, statistical analysis was performed combining both sexes, and significance was *p<0.05; data sets (mean±SEM) as analysed using two-way ANOVA with Bonferroni's multiple comparisons test. MPE, maximum possible effect.

FIG. 2. The effect of systemic subcutaneous administration of YM-254890 (YM) on spinal analgesia. (A) Dose-response effect of different concentrations of subcutaneous YM (0.1, 0.3, 0.5 and 1 mg/kg) and vehicle was tested on the tail immersion test after 30 min of administration. Treatment: F_{(1, 40)}=5.14; dose: F_{(3, 40)}=2.175; and interaction: F_{(3, 40)}=5.85. Two-way analysis of variance (ANOVA) with Bonferroni's post hoc test. (B) Effect of combined administration of subcutaneous YM (0.25 and 0.5 mg/kg) with a single dose of subcutaneous morphine (5 mg/kg) on the tail immersion test after 30 min of administration. YM was administered 10 min before the administration of morphine. Treatment: F_{(1, 20)}=8.21; dose: F_{(1, 20)}=1.38; and interaction: F_{(1, 20)}=1.75. Two-way ANOVA with Bonferroni's post hoc test. In all panels, statistical analysis was performed combining both sexes, and significance was *p<0.05; data sets (mean±SEM) as analysed using two-way ANOVA with Bonferroni's post hoc test. MPE, maximum possible effect.

FIG. 3. The effect of local intrathecal treatment of YM-254890 (YM) on spinal analgesia and locomotion. (A) Dose-response effect of different concentrations of intrathecal YM (0.5, 1.5, 3.0 and 4.5 nmol) and vehicle was tested on tail immersion test after 30 min of administration. Treatment: F_{(1, 40)}=81.67; dose: F_{(3, 40)}=22.93; and interaction: F_{(3, 40)}=12.19. Two-way analysis of variance (ANOVA) with Bonferroni's post hoc test. (B) Effect of combined administration of intrathecal YM (0.5, 1.5, 3.0 and 4.5 nmol) with a single low dose of subcutaneous morphine (2.5 mg/kg) on the tail immersion test after 30 min of administration. YM was administered 10 min before the administration of morphine. Treatment: F_{(1, 38)}=185.2; dose: F_{(3, 38)}=22.00; and interaction: F_{(3, 38)}=22.88. Two-way ANOVA with Bonferroni's post hoc test. (C) Changes in cumulative locomotor activity during 120 min of observation in open-field test by intrathecal administration of YM (3.0 nmol), subcutaneous morphine (2.5 mg/kg) and intrathecal YM (3.0 nmol, 10 min before morphine) with subcutaneous morphine (2.5 mg/kg)-treated mice. Treatment: F_{(1, 20)}=1.35; effect of morphine: F_{(1, 20)}=11.36; and interaction: F_{(1, 20)}=0.032. Two-way ANOVA with Bonferroni's post hoc test. In all panels, statistical analysis was performed combining both sexes, and significance was *p<0.05; data sets (mean±SEM) as analysed using two-way ANOVA with Bonferroni's multiple comparisons test. MPE, maximum possible effect.

FIG. 4. The effect of YM-254890 (YM) treatment on morphine-provoked inhibition of dorsal root ganglion (DRG) nociceptors. (A) Representative voltage traces from a continuous 0- to 2-nA ramp stimulation protocol illustrating excitability of a cultured DRG neuron at baseline, after bath application of either 1 μM morphine or 100 nM YM followed by both 100 nM YM+1 μM morphine. (B) Quantification of rheobase from DRG recordings illustrated in (A). Co-application of YM and morphine prevented action potential firing throughout the 2-nA ramp protocol in all recordings. YM: F_{(1, 24)}=6032; morphine: F_{(1, 24)}=7591; and interaction: F_{(1, 24)}=4208. Two-way analysis of variance (ANOVA) with Bonferroni's multiple comparisons test. (C) Quantification of resting input resistance at baseline and after bath application of either 1 μM morphine or 100 nM YM followed by both 100 nM YM+1 μM morphine. YM: F_{(1, 24)}=6.636; morphine: F_{(1, 24)}=172.7; and interaction: F_{(1, 24)}=57.75. Two-way ANOVA with Bonferroni's multiple comparisons test. Statistical analysis was performed combining both sexes, and significance was *p<0.05.

FIG. 5. YM-254890 (YM) and morphine interact to inhibit action potentials (APs) of dorsal root ganglion (DRG) nociceptors. (A) Representative current traces of AP profiles evoked by a 2-ms, 40-mV voltage step at baseline and after bath application of either 1 μM morphine or 100 nM YM followed by 100 nM YM+1 μM morphine. (B) Quantification of peak AP amplitude normalized to capacitance. Co-application of YM and morphine greatly inhibited evoked depolarization. YM: F_{(1, 20)}=283.0; morphine: F_{(1, 20)}=633.3; and interaction: F_{(1, 20)}=46.29. Two-way analysis of variance (ANOVA) with Bonferroni's multiple comparisons test. Statistical analysis was performed combining both sexes, and significance was *p<0.05.

FIG. 6. Graphs illustrating the effect of systemic subcutaneous administration of YM-254890 (YM) on nociception. (A) Dose-response effect of different concentration of subcutaneous YM and vehicle were tested on hot plate test for 3 h. (i) YM 0.1 mg/kg: Treatment F_{(1, 80)}=0.31, time F_{(7, 80)}=0.84, interaction F_{(7, 80)}=0.59. (ii) YM 0.3 mg/kg: Treatment F_{(1, 80)}=0.001, time F_{(7, 80)}=0.76, interaction F_{(7, 80)}=0.20. (iii) YM 0.5 mg/kg: Treatment F_{(1, 80)}=10.27, time F_{(7, 80)}=0.26, interaction F_{(7, 80)}=0.26. (iv) YM 1 mg/kg: Treatment F_{(1, 80)}=3.62, time F_{(7, 80)}=0.81, interaction F_{(7, 80)}=0.90. YM is labelled in each graph. Two-way ANOVA with Bonferroni's post hoc test. (B) Time course effect of combined administration of subcutaneous YM (0.25 and 0.5 mg/kg) with single dose of subcutaneous morphine (5 mg/kg) on hot plate test for 2 h. YM was administered 10 min before the administration of morphine. (i) YM 0.25 mg/kg: Treatment F_{(1, 90)}=46.32, time F_{(8, 90)}=44.07, interaction F_{(8, 90)}=2.44. (ii) YM 0.5 mg/kg: Treatment F_{(1, 90)}=177.8, time F_{(8, 90)}=19.30, interaction F_{(8, 90)}=2.62. Morphine+YM is labeled in each graph. Two-way ANOVA with Bonferroni's post hoc test. N=6 mice/group. In all panels statistical analysis was performed combining both sexes and significance was * p<0.05 and **p<0.001, data sets (mean±SEM) as analyzed using two-way ANOVA with Bonferroni's post hoc tests.

FIG. 7. Graphs illustrating the effect of intracerebroventricular injection of YM-254890 (YM) on nociception (A) Dose-response effect of different concentration of i.c.v. YM (0.4, 1.2, 4 nmol) and vehicle were tested on hot plate test after 10 min of administration. Treatment F_{(1, 30)}=45.04, dose F_{(2, 30)}=6.40, interaction F_{(2, 30)}=10.86. Two-way ANOVA with Bonferroni's post hoc test. (B) Time course effect of different concentration of i.c.v. YM and vehicle were tested on hot plate test for 2 h. (i) YM 0.4 nmol: Treatment F_{(1, 5)}=0.62, time F_{(7, 35)}=1.57, interaction F_{(7, 35)}=1.55. (ii) YM 1.2 nmol: Treatment F_{(1, 5)}=2.28, time F_{(7, 35)}=0.97, interaction F_{(7, 35)}=3.45. (iii) YM 4 nmol: Treatment F_{(1, 5)}=17.86, time F_{(7, 35)}=13.54, interaction F_{(7, 35)}=15.30. YM is labeled in each graph. Two-way ANOVA with Bonferroni's post hoc test. N=6 mice/group. In all panels statistical analysis was performed combining both sexes and significance was * p<0.05 and **p<0.001, data sets (mean±SEM) as analyzed using two-way ANOVA with Bonferroni's post hoc tests.

FIG. 8. Graphs illustrating the effect of systemic subcutaneous administration of YM-254890 (YM) on spinal analgesia. (A) Dose-response effect of different concentration of subcutaneous YM and vehicle were tested on tail immersion test for 2 h. (i) YM 0.1 mg/kg: Treatment F_{(1, 60)}=0.32, time F_{(5, 60)}=1.34, interaction F_{(5, 60)}=0.84. (ii) YM 0.3 mg/kg: Treatment F_{(1, 60)}=0.96, time F_{(5, 60)}=0.73, interaction F_{(5, 60)}=0.35. (iii) YM 0.5 mg/kg: Treatment F_{(1, 60)}=0.55, time F_{(5, 60)}=0.69, interaction F_{(5, 60)}=0.86. (iv) YM 1 mg/kg: Treatment F_{(1, 60)}=2.37, time F_{(5, 60)}=1.65, interaction F_{(5, 60)}=2.70. YM is labeled in each graph. Two-way ANOVA with Bonferroni's post hoc test. (B) Time course effect of combined administration of subcutaneous YM (0.25 and 0.5 mg/kg) with single dose of subcutaneous morphine (5 mg/kg) on tail immersion test for 2 h. YM was administered 10 min before the administration of morphine. (i) YM 0.25 mg/kg: Treatment F_{(1, 60)}=7.28, time F_{(5, 60)}=30.48, interaction F_{(5, 60)}=0.71. (ii) YM 0.5 mg/kg: Treatment F_{(1, 60)}=52.40, time F_{(5, 60)}=35.48, interaction F_{(5, 50)}=3.17. Morphine+YM is labeled in each graph. Two-way ANOVA with Bonferroni's post hoc test. N=6 mice/group. In all panels statistical analysis was performed combining both sexes and significance was *p<0.05, **p<0.001, ***p<0.0001 and ****p<0.00001, data sets (mean±SEM) as analyzed using two-way ANOVA with Bonferroni's post hoc tests.

FIG. 9A. Graphs illustrating the effect of local intrathecal administration of YM-254890 (YM) on spinal analgesia. (A) Dose-response effect of different concentration of intrathecal YM and vehicle were tested on tail immersion test for 2 h. (i) YM 0.5 nmol: Treatment F_{(1, 60)}=4.92, time F_{(5, 60)}=0.44, interaction F_{(5, 60)}=0.80. (ii) YM 1.5 nmol: Treatment F_{(1, 60)}=13.63, time F_{(5, 60)}=2.38, interaction F_{(5, 60)}=1.57. (iii) YM 3.0 nmol: Treatment F_{(1, 60)}=109.7, time F_{(5, 60)}=15.41, interaction F_{(5, 60)}=16.01. (iv) YM 4.5 nmol: Treatment F_{(1, 60)}=116.1, time F_{(5, 60)}=14.21, interaction F_{(5, 60)}=11.88. YM is labeled in each graph. Two-way ANOVA with Bonferroni's post hoc test. N=6 mice/group. In all panels statistical analysis was performed combining both sexes and significance was *p<0.05, **p<0.001, ***p<0.0001, and ****p<0.00001 data sets (mean±SEM) as analyzed using two-way ANOVA with Bonferroni's post hoc tests.

FIG. 9B. Graphs illustrating the effect of local intrathecal administration of YM-254890 (YM) on spinal analgesia. (B) Time course effect of combined administration of intrathecal YM with single dose of subcutaneous morphine (2.5 mg/kg) on tail immersion test for 2 h. YM was administered 10 min before the administration of morphine. (i) YM 0.5 nmol: Treatment F_{(1, 10)}=15.40, time F_{(5, 50)}=39.79, interaction F_{(5, 50)}=1.38. (ii) YM 1.5 nmol: Treatment F_{(1, 60)}=68.29, time F_{(5, 60)}=29.80, interaction F_{(5, 60)}=0.40. (iii) YM 3.0 nmol: Treatment F_{(1, 60)}=306.3, time F_{(5, 60)}=33.79, interaction F_{(5, 60)}=0.99. (iv) YM 4.5 nmol: Treatment F_{(1, 48)}=3103, time F_{(5, 48)}=40.06, interaction F_{(5, 48)}=11.70. Morphine+YM is labeled in each graph. Two-way ANOVA with Bonferroni's post hoc test. N=6 mice/group. In all panels statistical analysis was performed combining both sexes and significance was *p<0.05, **p<0.001, ***p<0.0001, and ****p<0.00001 data sets (mean±SEM) as analyzed using two-way ANOVA with Bonferroni's post hoc tests.

FIG. 10A-F. Physiological parameters of morphine-responsive DRG neurons (A) Illustration of Protocol 1, used to characterize hyperpolarization-activated current. (B) Representative trace of DRG produced by Protocol 1. Hyperpolarization-activated current delta is indicated. (C) Quantification of hyperpolarization-activated current of nociceptors. (D) Illustration of Protocol 2. (E) Representative trace produced by Protocol 2, used to characterize activation threshold and A-current inactivation rate Tau as indicated. (F) Quantification of nociceptor Acurrent tau.

FIG. 10G-I. Physiological parameters of morphine-responsive DRG neurons (G) Illustration of Protocol 3. (H) Representative trace produced by Protocol 3, used to determine the inactivation decay constant of the first inward current response as indicated, and the response amplitude. (I) Quantification of the inactivation decay constant.

FIG. 11. The effect of YM-254890 (YM) on Substance P induced excitation of DRG nociceptors. (A) Representative voltage traces from a continuous 0-2 nA ramp stimulation protocol illustrating excitability of cultured DRG neurons at baseline (black), and after bath application of 10 nM Substance P (SP, purple) followed by 10 nM SP and 100 nM YM (green). (B) Quantification of rheobase from DRG recordings illustrated in A. Treatment: F_{(8, 24)}=5.812. One-way repeatedmeasures ANOVA with Tukey's multiple comparisons test. (C) Quantification of resting membrane potential from DRG recordings illustrated in A. Treatment: F_{(8, 24)}=23.16. One-way repeatedmeasures ANOVA with Tukey's multiple comparisons test. Statistical analysis was performed combining both sexes, and significance was ***p<0.01, and ****p<0.0001.

### DETAILED DESCRIPTION AND EXAMPLES

### I. Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s), or method step(s) that do not materially change the basic and novel characteristics of the compositions and methods described herein may be included in those compositions or methods.

All ranges are to be interpreted as encompassing the endpoints in the absence of express exclusions, such as “not including the endpoints”; thus, for example, “within 10-15” includes the values 10 and 15. One skilled in the art will understand that the recited ranges include the end values, as whole numbers in between the end values, and where practical, rational numbers within the range (e.g., the range 5-10 includes 5, 6, 7, 8, 9, and 10, and where practical, values such as 6.8, 9.35, etc.). When the specification discloses a specific value for a parameter, the specification should be understood as alternatively disclosing the parameter at “about” that value. When values are expressed as approximations, by use of the antecedent “about,” it will be understood that the particular value forms a further aspect. For example, if the value “about 10” is disclosed, then “10” is also disclosed. The term “about” or “approximately” indicates within an acceptable error range for the particular value as determined by one of ordinary skill in the art, which will depend in part on how the value is measured or determined, i.e., the limitations of the measurement system. For example, “about” can mean within 1 standard deviation, per the practice in the art. Alternatively, “about” can mean a range of up to 0 to 20%, 0 to 10%, 0 to 5%, or up to 1% of a given value. Where particular values are described in the application and claims, unless otherwise stated the term “about” meaning within an acceptable error range for the particular value should be assumed.

An “active ingredient” is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. A dosage form for a pharmaceutical contains the active pharmaceutical ingredient, and optionally one or more excipients or other material that is pharmaceutically inert. During formulation development, the excipients can be selected so that the active ingredient can reach the target site in the body at the desired rate and extent.

An “analog” refers to a molecule that structurally resembles a reference molecule (e.g., YM-254890 or FR-900359) but which has been modified in a targeted and controlled manner, by replacing a specific substituent of the reference molecule with an alternate substituent. Compared to the reference molecule, an analog would be expected, by one skilled in the art, to exhibit the same or similar utility. Synthesis and screening of analogs to identify variants of known compounds having improved characteristics (such as higher binding affinity for a target molecule) is an approach that is well known in pharmaceutical chemistry.

A “derivative” of a first compound is a compound that has a three dimensional structure that is similar to at least a part of the first compound. In some embodiments, a derivative is a compound that is derived from, or imagined to derive from, another compound such as by substitution of one atom or group with another atom or group. In some embodiments, derivatives are compounds that at least theoretically can be formed from a common precursor compound.

A “pharmacologically effective amount,” “therapeutically effective amount,” or simply “effective amount” refers to that amount (dose) of a described active pharmaceutical ingredient or pharmaceutical composition to produce the intended pharmacological, therapeutic, or preventive result. An effective amount can also refer to the amount of, for example an excipient, in a pharmaceutical composition that is sufficient to achieve the desired property of the composition. An effective amount can be administered in one or more administrations, applications, or dosages.

As used herein, “dose,” “unit dose,” or “dosage” can refer to physically discrete units suitable for use in a subject, each unit containing a predetermined quantity of active pharmaceutical ingredient and/or a pharmaceutical composition thereof calculated to produce the desired response or responses in association with its administration.

The terms “treat,” “treatment,” and the like, mean the methods or steps taken to prevent or provide relief from or alleviation of the number, severity, and/or frequency of one or more symptoms of a disease or condition in a subject. Treating generally refers to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term treatment can include: (a) preventing the disease from occurring in a subject who may be predisposed to the disease but has not yet been diagnosed as having it; (b) inhibiting the disease, e.g., arresting its progression or development; and/or (c) relieving the disease, i.e., mitigating or ameliorating the disease and/or one or more of its symptoms or conditions. Treating can refer to both therapeutic treatment alone, prophylactic treatment alone, or both therapeutic and prophylactic treatment. Those in need of treatment (subjects in need thereof) can include those already with the disease, disorder, or condition or those in which disease, disorder, or condition is to be prevented. Treating can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the symptom without affecting or removing an underlying cause of the symptom.

“Opioid” includes opioid drugs and opioid-related drugs or compounds that are members of a class of drugs either derived from, or chemically similar to, compounds found in opium poppies. Examples of opioids include legal prescription painkillers like oxycodone (OxyContin®), hydrocodone (Vicodin®), morphine, codeine, dihydrocodeine, fentanyl, pethidine, hydromorphone, buprenorphine, and methadone and the like, and illegal drugs such as opium and heroin. Opioids also include antagonist drugs such as naloxone, and endogenous peptides such as endorphins. In some embodiments, opioid compounds can also include partial agonists of MOR, e.g., buprenorphine and methadone.

An “opioid use disorder” is a substance use disorder (persistent use of a drug despite harm and adverse consequences) relating to the use of an opioid. Signs of the disorder include a strong desire to use opioids, impaired control over its use, increased tolerance to opioids, persistent use despite harmful consequences, trouble reducing use, and withdrawal symptoms with discontinuation. Opioid withdrawal symptoms include, but are not limited to, nausea, muscle aches, diarrhea, trouble sleeping, agitation, and a low mood. Addiction and dependence are components of a substance use disorder.

Withdrawal, withdrawal symptoms, or withdrawal syndrome refers to a collection of symptoms and the degree of severity which the symptoms occur on cessation or abrupt reduction of use of a psychoactive substance (e.g., an opioid drug) that has been taken repeatedly, usually for a prolonged period and/or in high doses. The syndrome may be accompanied by signs of physiological and/or emotional disturbance. A withdrawal symptom is one of the indicators of a dependence syndrome.

The μ-opioid receptors are a class of opioid receptors with a high affinity for endogenous opioid peptides enkephalins and beta-endorphin, but a low affinity for dynorphins. They are also referred to as μ-opioid peptide (MOR) receptors. MOR is encoded by the OPRM gene. The prototypical exogenous MOR agonist is morphine, the primary psychoactive alkaloid in opium. MOR is an inhibitory G-protein coupled receptor that activates several inhibitory G protein subunits, including Giα, Goα, Gzα, and Gβγ (G beta-gamma), inhibiting activity of adenylate cyclase to lower cAMP levels and several ion channels to reduce neuronal excitability and synaptic transmission. Activation of the MOR by an agonist, such as morphine, causes analgesia, sedation, slightly reduced blood pressure, itching, nausea, euphoria, decreased respiration, miosis (constricted pupils), and decreased bowel motility often leading to constipation. Some of these effects, such as analgesia, sedation, euphoria, itching, and decreased respiration, tend to lessen with continued use as tolerance develops. As with other G protein-coupled receptors, signaling by the MOR is terminated through several different mechanisms, which are upregulated with chronic use, leading to rapid tachyphylaxis.

A “Gα_{q/11} inhibitor” is a compound that down-regulates or inhibits Gα_{q/11} (i.e., GNAQ and/or GNA11) expression, Gα_{q/11} (i.e., GNAQ and/or GNA11) mRNA levels, or cellular activity of Gα_{q/11}, or down-regulates or inhibits Gα_{q/11} pathway activity. The Gα_{q/11} is a group of two proteins that comprise Gqα (encoded by the GNAQ gene (Gene ID: 2776, GenBank AF493896.1 (ORF), NCBI Reference Sequence NM_002072.5 mRNA) and Gα₁₁ (encoded by the GNA11 gene, Gene ID 2767, GenBank CR457004.1 (ORF), NCBI Reference Sequence NM_002067.5)) subunits.

Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “complementarity” refers to the ability of a polynucleotide to form hydrogen bond(s) (hybridize) with another polynucleotide sequence by either traditional Watson-Crick or other non-traditional types. A percent complementarity indicates the percentage of bases, in a contiguous strand, in a first nucleic acid sequence which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). Percent complementarity is calculated in a similar manner to percent identity.

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339:823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT, NGAN, NGCG, NGAG, NGNG, NGC, and NGA.

A “trans-activating CRISPR RNA” (tracrRNA) is an RNA species facilitates binding of the RNA-guided DNA endonuclease (e.g., Cas) to the guide RNA.

A “CRISPR system” comprises a guide RNA, either as a crRNA and a tracrRNA (dual guide RNA) or an sgRNA, and an RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

A “subject” can be an animal. The animal can be a mammal. A mammal can be, but is not limited to a human. The subject can have experienced and/or exhibited at least one symptom of depression or stress-induced depression. The subject can have been diagnosed with at least one symptom of depression or stress-induced depression. The subject can be experiencing or exhibiting at least one symptom of depression or stress-induced depression. The subject can be at risk of experiencing or exhibiting at least one symptom of depression or stress-induced depression In some embodiments, the at least one symptom, is caused by, has been cause by, or is likely caused by stress. The depression can be, but is not limited to, a major depressive disorder or episode.

### II. Gα_{q/11} Inhibitor

Cyclic depsipeptides YM-254890 (YM) and FR-900359 (FR) have been identified as selective Gog/11 inhibitors. Their actions have been well characterized mechanistically (Nishimura A et al. “Structural basis for the specific inhibition of heterotrimeric Gq protein by a small molecule.” Proceedings of the National Academy of Sciences (2010) 107:13666-13671). Their in vivo efficacy has been tested for cardiovascular effects, but they have not previously evaluated for the effects in the nervous system.

YM-254890 (Cas No. 568580-02-9). YM-254890 is a selective Gα_{q/11} inhibitor isolated from chromobacterium species (Taniguchi M et al. “YM-254890, a novel platelet aggregation inhibitor produced by Chromobacterium sp. QS3666.” J Antibiot (Tokyo) (2003) 56:358-363).

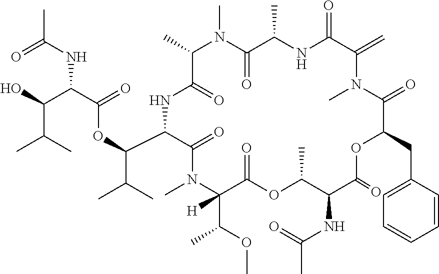 [SMILES: C=C1C(=O)N[C@@H](C)C(=O)N(C)[C@@H](C)C(=O)N[C@@H]([C@H](OC(=O)[C@@H](NC(C)=O)[C@H](O)C(C)C)C(C)C)C(=O)N(C)[C@@H]([C@@H](C)OC)C(=O)O[C@H](C)[C@H](NC(C)=O)C(=O)O[C@H](Cc2ccccc2)C(=O)N1C]

FR-900359 (Cas No. 107530-18-7)

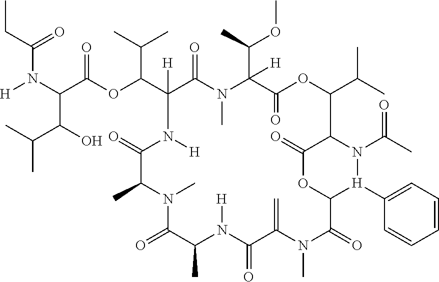

In addition, small molecule inhibitors of Gα_{q/11} have also been identified. Such small molecule inhibitors of Gα_{q/11} include, but are not limited to, GQ262 and GQ127.

### GQ262

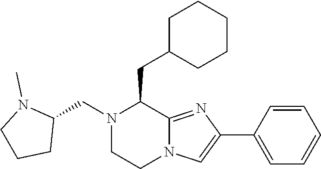 [SMILES: CN1CCC[C@H]1CN1CCn2cc(-c3ccccc3)nc2[C@@H]1CC1CCCCC1]

### GQ127: (2S,3R)-2-amino-1-((S)-8-(cyclohexylmethyl)-2-phenyl-5,6-dihydroimidazo[1,2-a]pyrazin-7 (8H)-yl)-3-methylpentan-1-one

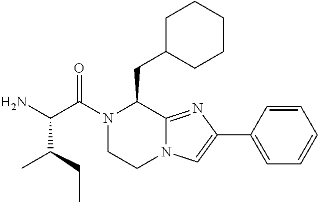 [SMILES: CC[C@@H](C)[C@H](N)C(=O)N1CCn2cc(-c3ccccc3)nc2[C@@H]1CC1CCCCC1]

A nucleic acid-based inhibitor comprises any polynucleotide that is not translated into protein but whose presence in the cell decreases expression of a target gene. A nucleic acid-based inhibitor comprises a polynucleotide, including polynucleotides containing nucleotide analogs, containing a sequence whose presence or expression in a cell causes the degradation of or inhibits the function or translation of a specific cellular RNA, usually an mRNA, in a sequence-specific manner. Inhibition of RNA can thus effectively inhibit expression of a gene from which the RNA is transcribed. Nucleic acid-based inhibitors are selected from the group comprising: RNA interference (RNAi) polynucleotides, siRNA, microRNA, dsRNA, RNA Polymerase II transcribed DNAs encoding siRNA or antisense oligonucleotides, RNA Polymerase III transcribed DNAs encoding siRNA or antisense oligonucleotides, ribozymes, antisense oligonucleotides, and antisense nucleic acid, which may be RNA, DNA, or artificial nucleic acid.

An RNA interference (RNAi) polynucleotide is a molecule capable of inducing RNA interference through interaction with the RNA interference pathway machinery of mammalian cells to degrade or inhibit translation of messenger RNA (mRNA) transcripts of a transgene a sequence specific manner. RNAi polynucleotides may be selected from the group comprising: siRNA, microRNA, double-strand RNA (dsRNA), short hairpin RNA (shRNA), and expression cassettes encoding RNA capable of inducing RNA interference. siRNA comprises a double stranded structure typically containing 15-50 base pairs and preferably 21-25 base pairs and having a nucleotide sequence identical (perfectly complementary) or nearly identical (partially complementary) to a sequence present in an expressed target gene or RNA within the cell. An siRNA may have overhangs, such as dinucleotide 3′ overhangs. An siRNA may be composed of two annealed polynucleotides or a single polynucleotide that forms a hairpin structure. An siRNA molecule comprises a sense region and an antisense region. In one embodiment, the siRNA of the conjugate is assembled from two oligonucleotide fragments wherein one fragment comprises the nucleotide sequence of the antisense strand of the siRNA molecule and a second fragment comprises nucleotide sequence of the sense region of the siRNA molecule. In another embodiment, the sense strand is connected to the antisense strand via a linker molecule, such as a polynucleotide linker or a non-nucleotide linker.

MicroRNAs (miRNAs) are small noncoding RNA gene products about 22 nucleotides long that direct destruction or translational repression of their mRNA targets. For miRNAs, the complex binds to target sites usually located in the 3′ UTR of mRNAs that typically share only partial homology with the miRNA. A “seed region”—a stretch of about seven (7) consecutive nucleotides on the 5′ end of the miRNA that forms perfect base pairing with its target—plays a key role in miRNA specificity. Binding of the RISC/miRNA complex to the mRNA can lead to either the repression of protein translation or cleavage and degradation of the mRNA. Recent data indicate that mRNA cleavage happens preferentially if there is perfect homology along the whole length of the miRNA and its target instead of showing perfect base-pairing only in the seed region.

An antisense oligonucleotide (ASOs) comprises an oligonucleotide having a nucleobase sequence that is complementary to a target nucleic acid or region or segment thereof. Antisense oligonucleotides include, but are not limited to: morpholinos, 2′-O-methyl polynucleotides, DNA, RNA, and the like. In some embodiments, an antisense oligonucleotide is specifically hybridizable to a target nucleic acid or region or segment thereof. ASOs can comprise nucleobase sequence and optionally one or more additional features, such as a conjugate group or terminal group. ASOs may be single-stranded and double-stranded compounds. ASOs include, but are not limited to, oligonucleotides, ribozymes, gapmers, and morpholinos (peptide-conjugated phosphorodiamidate oligonucleotides (PPMOs) or simply phosphorodiamidate oligonucleotides (PMOs)). Antisense nucleic acids act by hybridization of an antisense nucleotide sequence to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target. Antisense nucleic acid can inhibit gene expression by reducing the levels of target RNA in a cell or by inhibiting translation, splicing, or activity of an RNA in a cell.

Gapmers are short DNA antisense oligonucleotide structures with RNA-like segments on both sides of the sequence. Gapmers are designed to hybridize to sequence in a target RNA and inhibit expression of the gene through the induction of RNase H cleavage. Binding of the gapmer to the target has a higher affinity due to the modified RNA flanking regions. The RNA flanking regions can have modified RNA nucleotides, such as, but not limited to, 2′-MOE (2′-O-(2-Methoxyethyl) modified nucleotides, or LNA (locked nucleic acid) modified nucleotides.

A nucleic acid-based inhibitor may be polymerized in vitro, recombinant, contain chimeric sequences, or derivatives of these groups. The nucleic acid-based inhibitor may contain ribonucleotides, deoxyribonucleotides, synthetic nucleotides, or any suitable combination such that the target RNA and/or gene is inhibited. In addition, these forms of nucleic acid may be single, double, triple, or quadruple stranded.

A nucleic acid-based inhibitor may be expressed from a DNA vector in a cell. Nucleic acid-based inhibitor expression cassettes can be transcribed in the cell to produce hairpin RNAs (including shRNA or miRNA), separate sense and anti-sense strand linear siRNAs, an antisense nucleic acid, or a ribozyme. Coding sequence for the nucleic acid-based inhibitor can be operatively linked to a promoter for expression of the nucleic acid-based inhibitor in a cell. The promoter can be an RNA polymerase III promoter of an RNA polymerase II promoter. A RNA polymerase III promoter can be, but is not limited to, a U6 promoter, a H1 promoter, or a tRNA promoter. A RNA polymerase II promoter can be, but is not limited to, a U1 promoter, a U2 promoter, a U4 promoter, a U5 promoter, an snRNA promoter, a microRNA promoter, or a mRNA promoter.

In some embodiments, a Gα_{q/11} nucleic acid-based inhibitor comprises a double stranded ribonucleic acid (dsRNA) agent, such as an siRNA, for inhibiting expression of a GNAQ gene and/or a GNA11 gene in a cell, wherein the dsRNA agent comprises a sense strand and an antisense strand, wherein the sense strand comprises at least 15 contiguous nucleotides differing by no more than 3 nucleotides from a sequence present in a GNA [symbol] and/or GNA11 mRNA and the antisense strand comprises at least 15 contiguous nucleotides differing by no more than 3 nucleotides from a sequence present in a complement of a GNA [symbol] and/or GNA11 mRNA.

In some embodiments, a Gα_{q/11} nucleic acid-based inhibitor comprises a double stranded ribonucleic acid (dsRNA) agent, such as an siRNA, for inhibiting expression of a GNA [symbol] and/or GNA11 gene in a cell, wherein the dsRNA agent comprises a sense strand and an antisense strand, wherein the sense strand comprises at least 17, at least 18, at least 19, or at least 20 contiguous nucleotides from the nucleotide sequence of the GNA [symbol] and/or GNA11 mRNA and the antisense strand comprises least 17, at least 18, at least 19, or at least 20 contiguous nucleotides from a complement of the GNA [symbol] and/or GNA11 mRNA (i.e., at least 17, at least 18, at least 19, or at least 20 contiguous nucleotides that are complementary to the sense strand).

In some embodiments, a Gα_{q/11} nucleic acid-based inhibitor comprises a double stranded ribonucleic acid (dsRNA) agent, such as an siRNA, for inhibiting expression of a GNA [symbol] and/or GNA11 gene in a cell, wherein the dsRNA agent comprises a sense strand and an antisense strand, wherein the sense strand comprises 17, 18, 19, 20, 21, 22, 23, 24, or 25 contiguous nucleotides from the nucleotide sequence of the GNA [symbol] and/or GNA11 mRNA and the antisense strand comprises least 17, 18, 19, 20, 21, 22, 23, 24, or 25 contiguous nucleotides from a complement of the GNA [symbol] and/or GNA11 mRNA (i.e., 17, 18, 19, 20, 21, 22, 23, 24, or 25 contiguous nucleotides that are complementary to the sense strand).

In some embodiments, a Gα_{q/11} nucleic acid-based inhibitor comprises a dsRNA agent, such as an siRNA, for inhibiting expression of a the GNA [symbol] gene in a cell. In some embodiments, a Gα_{q/11} nucleic acid-based inhibitor comprises a dsRNA agent, such as an siRNA, for inhibiting expression of the GNA11 gene in a cell. In some embodiments, a Gog/11 nucleic acid-based inhibitor comprises dsRNA agents, such as siRNAs, for inhibiting expression of the GNA [symbol] gene and the GNA11 gene in a cell.

ASOs can be designed using methods available in the art. In some embodiments, a Gα_{q/11} nucleic acid-based inhibitor comprises an ASO for inhibiting expression of a GNA [symbol] and/or GNA11 gene in a cell, wherein the ASO comprises at least 10 contiguous nucleotides differing by no more than 3 nucleotides from a complement of the nucleotide sequence of the GNAQ and/or GNA11 mRNA. In some embodiments, the ASO comprises a gapmer.

In some embodiments, a Gα_{q/11} nucleic acid-based inhibitor comprises an ASO for inhibiting expression of a GNA [symbol] and/or a GNA11 gene in a cell, wherein the ASO comprises 10-20 contiguous nucleotides differing by no more than 3 nucleotides from a sequence present in a complement of a of the GNA [symbol] and/or GNA11 mRNA. In some embodiments, the ASO comprises a gapmer.

In some embodiments, a Gog/11 nucleic acid-based inhibitor comprises an ASO for inhibiting expression of a of the GNA [symbol] and/or GNA11 gene in a cell, wherein the ASO comprises at least 10, 11, 12, 13, 14, 15, 16, 17, 18, 19, or 20 contiguous nucleotides from a sequence present in a complement of the GNA [symbol] and/or GNA11 mRNA. In some embodiments, the ASO comprises a gapmer.

In some embodiments, a Gα_{q/11} nucleic acid-based inhibitor comprises an ASO for inhibiting expression of a of the GNA [symbol] and/or GNA11 gene in a cell, wherein the ASO comprises at 16, 17, 18, 19, or 20 contiguous nucleotides from a sequence present in a complement of the GNA [symbol] and/or GNA1 mRNA. In some embodiments, the ASO comprises a gapmer.

In some embodiments, a Gα_{q/11} nucleic acid-based inhibitor comprises an ASO for inhibiting expression of the GNA [symbol] gene in a cell. In some embodiments, a Gα_{q/11} nucleic acid-based inhibitor comprises an ASO for inhibiting expression of the GNA11 gene in a cell. In some embodiments, a Gα_{q/11} nucleic acid-based inhibitor comprises ASOs for inhibiting expression of the GNA [symbol] gene and the GNA11 gene in a cell.

In some embodiments, the Gα_{q/11} inhibitor comprises a Gα_{q/11} CRISPR-based system. Gα_{q/11} CRISPR systems can be used to modify, disrupt, or mutate the GNA [symbol] and/or GNA11 gene in a cell. A CRISPR system comprises an RNA-guided DNA endonuclease enzyme and a CRISPR RNA. In some embodiments, a CRISPR RNA is part of a guide RNA. In some embodiments, the RNA-guided DNA endonuclease enzyme is a Cas9 protein. Other RNA-guided DNA endonuclease enzymes can be used, including derivatives of Cas9 used for gene editing. In some embodiments, a CRISPR system comprises one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the of the GNA [symbol] and/or GNA11 gene genomic region. The Cas protein can be introduced into the cell in the form of a protein or a nucleic acid (DNA or RNA) encoding the Cas protein (e.g., operably linked to a promoter expressible in the cell). The guide RNA can be introduced into the cell in the form of RNA or a DNA encoding the guide RNA (e.g., operably linked to a promoter expressible in the cell). In some embodiments, the Gα_{q/11} CRISPR system can be delivered to a cell via a viral vector.

A Gα_{q/11} CRISPR system is designed to target the of the GNA [symbol] and/or the GNA11 gene. The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

A suitable guide sequence includes a 17-20 nucleotide sequence complementary to a target sequence in the GNA [symbol] and/or the GNA11 gene that is unique compared to the rest of the genome and immediately adjacent (5′) to a protospacer-adjacent motif (PAM) site. For the RNA-guided DNA endonuclease enzyme zCas9, a PAM site is NGG. Thus, any unique 17-20 nucleotide sequence immediately 5′ of a 5′-NGG-3′ in the of the GNA [symbol] and/or GNA11 gene or a complement thereof can be used in forming a gRNA. In some embodiments, the guide sequence is 100% complementary to the target sequence. In some embodiments, the guide sequence is at least 90% or at least 95% complementary to the target sequence. In some embodiments, the guide sequence contains 1 or 2 mismatches when hybridized to the target sequence. In some embodiments, a mismatch, if present, is located distal to the PAM, in the 5′ end of the guide sequence

In some embodiments, a Gα_{q/11} CRISPR system is designed to target the GNA [symbol] and/or the GNA11 gene in a cell. In some embodiments, a Gα_{q/11} CRISPR system is designed to target the GNA [symbol] gene in a cell. In some embodiments, a Gα_{q/11} CRISPR system is designed to target the GNA11 gene in a cell. In some embodiments, a Gα_{q/11} CRISPR system is designed to target the GNA [symbol] gene and the GNA11 gene in a cell.

### III. Formulations and Pharmaceutical Compositions

The Gα_{q/11} inhibitors can be formulated with one or more pharmaceutically acceptable excipients (including vehicles, carriers, diluents, and/or delivery polymers), thereby forming a pharmaceutical composition or medicament suitable for in vivo delivery to a subject, such as a human.

A pharmaceutical composition or medicament includes a pharmacologically effective amount of a Gα_{q/11} inhibitor and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support, or enhance stability, bioavailability or patient acceptability of the API, c) assist in product identification, and/or d) enhance any other attribute of the overall safety and effectiveness of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

A carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants, such as preservatives, wetting agents, emulsifying agents, and dispersing agents. A carrier may also contain isotonic agents, such as sugars, polyalcohols, sodium chloride, and the like into the compositions.

The pharmaceutical compositions can contain other additional components commonly found in pharmaceutical compositions. Such additional components can include, but are not limited to: anti-pruritics, astringents, local anesthetics, or anti-inflammatory agents (e.g., antihistamine, diphenhydramine, etc.).

The phrase “pharmaceutically acceptable” indicates that the substance or composition must be compatible chemically and/or toxicologically, with the other ingredients comprising a formulation, and/or the mammal being treated therewith. Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals, and more particularly in humans.

In some embodiments, the pharmaceutical compositions further comprise one or more additional active ingredients. The additional active pharmaceutical ingredients can be, but are not limited to, an opiate, an analgesic, an NSAID, acetaminophen, an antipsychotic, a mood stabilizer, and an antidepressant.

In some embodiments, a pharmaceutical compositions comprises a Gα_{q/11} inhibitor and an opiate. In some embodiments, a pharmaceutical compositions comprises a Gα_{q/11} inhibitor and a suboptimal or low dose of opiate. A suboptimal or low dose of an opiate is a dose of the opiate that is less than would typically be administered to a subject to produce a desired level of analgesia.

In some embodiments, a Gα_{q/11} inhibitor is formulated for use in combination with a opiate.

A Gα_{q/11} inhibitor or pharmaceutical compositions containing a Gα_{q/11} inhibitor can be formulated as a liquid formulation or as a solid formulation (including a powder or lyophilized formulation). The Gα_{q/11} inhibitor or pharmaceutical compositions containing a Gα_{q/11} inhibitor can be formulated as a capsule or tablet, a time-release capsule or tablet, a powder, granules, a solution, a suspension in an aqueous liquid or non-aqueous liquid, an oil-in-water emulsion, or as a water-in-oil liquid emulsion. The Gα_{q/11} inhibitor or pharmaceutical compositions containing a Gα_{q/11} inhibitor can be formulated for oral administration, aerosol or inhalation administration, nasal administration, injection, infusion, topical administration, rectal administration, transmucosal administration, transdermal administration, intravenous administration, intradermal administration, subcutaneous administration, intramuscular administration, intrathecal administration, or intraperitoneal administration.

A Gα_{q/11} inhibitor or pharmaceutical compositions containing a Gα_{q/11} inhibitor can be formulated or packaged in single-dose or multi-dose format. In some embodiments, a Gα_{q/11} inhibitor or a pharmaceutical composition containing a Gα_{q/11} inhibitor can be formulated for repeat dosing.

In some embodiments, kits containing a Gα_{q/11} inhibitor or a pharmaceutical composition containing a Gα_{q/11} inhibitor are described. In some embodiments, a kit comprising a Gα_{q/11} inhibitor or a pharmaceutical composition containing a Gα_{q/11} inhibitor further comprises instructions for use. Instructions include documents describing relevant materials or methodologies pertaining to the kit. The instructions may include one or more of: background information, list of components and their availability information (purchase information, etc.), brief or detailed protocols for using the kit, trouble-shooting guidance, references, technical support, indications, usage, dosage, administration, contraindications, and/or warnings concerning the use of the drug, and any other related documents. Instructions can be supplied with the kit or as a separate member component, either as a paper form or an electronic form. The instructions may include a notice in a form prescribed by a governmental agency regulating the manufacture, use or sale of pharmaceuticals or biological products, which notice reflects approval by the agency of manufacture, use or sale for human administration.

In some embodiments, a kit comprises two or more components, including at least one active pharmaceutical ingredient (i.e., a Gα_{q/11} inhibitor) and one or more inactive ingredients, excipient, diluents, and the like, and optionally instructions for preparation of the dosage form by the patient or person administering the drug to the patient. In some embodiments, a kit may further comprise optional components that aid in the administration of the unit dose to a subject, including but not limited to: vials for reconstituting powder forms, syringes for injection, customized IV delivery systems, inhalers, etc. Additionally, a kit can contain instructions for preparation and administration of the compositions. The kit can be manufactured as a single use unit dose for one subject, multiple uses for a particular subject (at a constant dose or in which the individual compounds may vary in potency as therapy progresses); or the kit may contain multiple doses suitable for administration to multiple subjects (“bulk packaging”). The kit components may be assembled in cartons, blister packs, bottles, tubes, and the like.

In some embodiments, a kit further includes an additional therapeutic agent. The additional therapeutic agent can be, but is not limited to, an opioid.

### IV. Use of Gα_{q/11} Inhibitor in Analgesia

Described are methods of inducing antinociception in treatment of pain comprising administering to a subject a Gα_{q/11} inhibitor. In some embodiments, a Gα_{q/11} inhibitor is administered to a subject to produce analgesia.

Described are methods of treating or reducing perception of pain comprising administering to a subject a pharmaceutically effective dose of a Gα_{q/11} inhibitor. The Gα_{q/11} inhibitor can be administered alone or in combination with a second analgesic. The second analgesic can be, but is not limited to, an opioid.

Described are methods of suppressing excitability of DRG nociceptors in a subject comprising, administering to the subject a Gα_{q/11} inhibitor or a Gα_{q/11} inhibitor and an opioid analgesic. Combining a Gα_{q/11} inhibitor and an opioid analgesic can provide a synergistic effect, completely or nearly completely suppressing DRG nociceptor firing. Suppressing excitability of DRG nociceptors can be used to block or suppress reception of noxious stimuli or perception of pain.

In some embodiments, methods of treating pain are described, the methods comprising administering to a subject a pharmaceutically effective dose of one or more Gα_{q/11} inhibitors. In some embodiments, methods or treating pain are described, the methods comprising administering to a subject a pharmaceutically effective dose of one or more Gα_{q/11} inhibitors and one or more opioid analgesics.

Opioid analgesics offer pain management but have substantial abuse liability. Increasing efficacy of opioid drugs can provide for decreased dosage, leading to decreased abuse liability. Increasing efficacy of opioid drugs can also provide for increase efficacy of pain treatment. Described are methods of increasing opioid efficacy (e.g., enhancing analgesic effect) or safety by administering to a subject a Gα_{q/11} inhibitor.

In some embodiments, methods for increasing analgesic response in subjects taking opioid related drugs and/or diminishing the dependence-causing liability of the opioid drug are provided. The methods comprise, administering to the subject Gα_{q/11} inhibitor. The subject can be, but is not limited to, a subject that is an acute user or a chronic user of opioids.

In some embodiments, administering a Gα_{q/11} inhibitor to a subject enhances analgesic response of the subject to an opioid. In some embodiments, administering a Gα_{q/11} inhibitor to a subject increases sensitivity of the subject to an opioid. Enhancing analgesic response to an opioid or enhancing sensitivity to an opioid includes, but is not limited to enhancing maximal response to the opioid, increasing response to a given dose of opioid, decreasing the amount of the opioid required to provide an effective analgesic response, increasing duration of analgesic response to the opioid, and/or decreasing tolerance of the opioid. A Gα_{q/11} inhibitor can be administered to a subject to enhance analgesic response of the subject to an opioid prior to an initial dose of the opioid, at the same time as an initial dose of the opioid, at the same time as one or more subsequent doses of the opioid, or after an initial dose of the opioid.

In some embodiments, administering a Gα_{q/11} inhibitor to a subject diminishes the dependence-causing liability of the opioid. Diminishes the dependence-causing liability of the opioid includes, but is not limited to, decreasing the amount of the opioid required to provide an effective analgesic, decreasing tolerance of the opioid, increasing safety of an opioid therapeutic, and/or decreasing risk of addiction or dependence on the opioid. In some embodiments, a Gα_{q/11} inhibitor is administered to a subject in combination with an opioid analgesic to mitigate one or more side-effects associated with opioid therapies, thereby increasing the safety of the opioid analgesic.

In some embodiments, a subject in need of opioid treatment, is administered a Gα_{q/11} inhibitor in addition to the opioid. The Gα_{q/11} inhibitor can be administered prior to administration of the opioid, concurrent with opioid administration, or subsequent to opioid administration. The Gα_{q/11} inhibitor may be administered to the subject to improve pain relief associated with opioid treatment. The Gα_{q/11} inhibitor can be administered to a subject prior to an initial dose of the opioid, at the same time as an initial dose of the opioid, at the same time as one or more subsequent doses of the opioid, or after an initial dose of the opioid. The subject can be, but is not limited to, a human.

Subjects taking any opioid related drugs are amenable to treatment with a Gα_{q/11} inhibitor. The opioid drugs include, but are not limited to, morphine, synthetic opioid related compounds, methadone, oxycodone, hydrocodone, codeine, dihydrocodeine, pethidine, hydromorphone, heroin, opium, and fentanyl.

The Gα_{q/11} inhibitor for use in the described methods is administered to a subject in an amount that is sufficient to achieve the desired therapeutic effect in the subject. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors, including the activity of the particular Gα_{q/11} inhibitor employed, the route of administration, the time of administration, or the rate of excretion of the particular compound being employed. Dosage can also depend on the duration of the treatment, or other drugs, compounds, and/or materials used in combination with the Gα_{q/11} inhibitor. Age, gender, weight, condition, general health, and prior medical history of the subject being treated can also affect dosage. Methods for determining optimal dosages are described in the art, e.g., Remington: “The Science and Practice of Pharmacy,” Mack Publishing Co., 20th ed., 2000.

A Gα_{q/11} inhibitor or pharmaceutical composition comprising a Gα_{q/11} inhibitor can be administered by a variety of methods known in the art. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations or compositions to administer such compositions to subjects. Any appropriate route of administration may be employed, for example, but not limited to, oral administration, intravenous, parenteral, transcutaneous, subcutaneous, and intramuscular administration.

A Gα_{q/11} inhibitor can be administered to a subject as a bolus administration, as an infusion, or as multiple administrations. A Gα_{q/11} inhibitor can be administered to a subject by intravenous injection, subcutaneous injection, local injection, epidural injection, intrathecal injection, or other administration routes suitable for administration of analgesic therapeutics. In some embodiments, the Gα_{q/11} inhibitor is administered local, as in, for example, intrathecal injection or epidural injection.

In some embodiments, the Gα_{q/11} inhibitor YM-254890 is administered intrathecally. Intrathecal administration of YM-254890 can produce analgesia in a dose-dependent manner with reduced side effects or without noticeable side effects.

A Gα_{q/11} inhibitor or a pharmaceutical composition comprising a Gα_{q/11} inhibitor can be administered to a subject once per day, more than once a day, for example, 2, 3, 4, 5, or 6 times a day, or as needed.

In some embodiments, epidural or intrathecal Gα_{q/11} inhibitor administration enhances the analgesic effect of an opioid drug. In some embodiments, Gα_{q/11} inhibitor administration enhances the analgesic effect of low dose opioid drug. In some embodiments, local Gα_{q/11} inhibitor administration enhances the analgesic effect of low dose opioid drug. In some embodiments, epidural or intrathecal Gα_{q/11} inhibitor administration enhances the analgesic effect of low dose opioid drug. In some embodiments, systemic, local, epidural, or intrathecal Gα_{q/11} inhibitor administration enhances efficacy of opioid drug analgesic effect. In some embodiments, systemic, local, epidural, or intrathecal Gα_{q/11} inhibitor administration enhances the level of opioid drug analgesic effect. In some embodiments, systemic, local, epidural, or intrathecal Gα_{q/11} inhibitor administration enhances the duration of opioid drug analgesic effect. Without wish to be bound by theory, Gα_{q/11} signalling may contribute to setting nociceptive thresholds, and may intersect with the receptor signalling pathways, e.g., MOR, involved in analgesia.

A pharmaceutical composition containing a Gα_{q/11} inhibitor and other therapeutic agents described herein (e.g., an opioid drug) can be administered by a variety of methods known in the art. The routes and/or modes of administration vary depending upon the desired results. Depending on the route of administration, the active therapeutic agent may be coated in a material to protect the compound from the action of acids and other natural conditions that may inactivate the agent. Conventional pharmaceutical practice may be employed to provide suitable formulations or compositions to administer such compositions to subjects. Any appropriate route of administration may be employed, for example, but not limited to, oral administration, intravenous, parenteral, transcutaneous, subcutaneous, and intramuscular administration.

It is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

### EXAMPLES

### Example 1. Experimental Procedures

A. Experimental animals. C57BL/6 mice (6-8 weeks old) of both sexes were used. Mice were housed in groups on a 12-h light-dark cycle (6:00 am light cycle; 6:00 pm dark cycle) with food (Teklad Global 16% protein rodent diets; Envigo Inc., WI, USA) and water available ad libitum. Animal groups were compiled to ensure minimization of factors (e.g., weight, sex, and health). Mice were within 20-28 g in weight at start of all studies. All tested groups contained a control group and consisted of male and female mice. Males and females were tested on the same day but at different times so that they were not in the room at the same time. All the experiments were tested during the light cycle between 8:00 am and 3:00 pm to avoid any long-term disruption of sleep cycles. Mice were transferred to the behavioural testing room at least 45 min before the first test to acclimatize. Following behavioural evaluation, animals were killed by CO₂ inhalation, followed by cervical dislocation and postmortem decapitation.

B. Drug treatments. YM-254890 (YM) (YM-254890 was purchased from AdipoGen® Life Sciences. The morphine used was sterile and free of preservatives (morphine sulphate. For all in vivo studies, the vehicle used for YM-254890 compound was 0.05% dimethyl sulfoxide (DMSO) in 5% dextrose solution unless normal saline (NaCl 0.9%) was indicated as for morphine. To observe the systemic effects of YM-254890 administration, YM-254890 doses (0.1, 0.3, 0.5 and 1 mg·kg−1) were given subcutaneously. The dose of YM-254890 for intrathecal injection was selected from pilot studies (data not shown) and represents the highest dose and volume up to which analgesic response could was recorded without causing significant side effects (i.e. respiratory depression and decreased blood pressure). The dose of YM-254890 for subcutaneous injection was taken from previous published studies. The dose of morphine selected for subcutaneous administration (2.5, 5 and 10 mg kg-1) is commonly used in mouse analgesia. For the combined administration of morphine and YM-254890 on thermal anti-nociception, YM-254890 was administered 10 min before the morphine. The latency of response was measured before injection of drugs (baseline latency response) and at different time intervals (post-treatment latency response) after drugs injection.

C. Intrathecal injections. Intrathecal (i.t.) administration was performed following the method described previously (Hylden J L et al. “Intrathecal morphine in mice: a new technique.” Eur J Pharmacol (1980) 67:313-316; Li D et al. “Direct Intrathecal Injection of Recombinant Adeno-associated Viruses in Adult Mice.” J Vis Exp (2019)) with slight modifications. All intrathecal injections were performed using a 25 μL Hamilton syringe with a 30-gauge needle. The injection volume was 5 μL for each mouse. Data suggests that this is likely to be the upper limit that can be reliably injected into the mouse without any appreciable redistribution of the drugs through the cerebrospinal fluid (CSF) to the basal cisterns of the brain. Each solution was injected without injection cannulae. Intrathecal injections were made into the L5-L6 intervertebral space of unanesthetized mice. The flick of the tail was considered indicative of a successful i.t. administration.

D. Intracerebral injections. Intracerebroventricular administration was performed following the method described previously with slight modifications (Haley T J et al. “Pharmacological effects produced by intracerebral injection of drugs in the conscious mouse.” British Journal of Pharmacology and Chemotherapy, (1957) 12:12-15; Narita M et al. “Reduced expression of a novel μ-opioid receptor (MOR) subtype MOR-1B in CXBK mice: Implications of MOR-1B in the expression of MOR-mediated responses.” The European Journal of Neuroscience (2003) 18:3193-3198)). On the day of the drug/vehicle injection or on the day of behavioural test, the mouse was secured at the nape of the neck and head by the investigator's thumb and forefinger, and head of the mouse was held against a V-shaped holder. A 27-gauge hypodermic needle attached with 25 μL. Hamilton microsyringe was inserted perpendicularly into the unilateral injection site into the hole with the depth of 3.5 mm. The injection volume was 4 μL.

### E. Behavior Assessment.

(1) Hot Plate test. Animals were tested for the assessment of anti-nociception and analgesic effects of morphine using a hotplate set to 52.5° C. The assay was performed as described previously (Bannon A W et al. “Models of nociception: Hotplate, tail-flick, and formalin tests in rodents.” In Current Protocols in Neuroscience, Chapter 8: Unit 8.9. Wiley (2007); Wang D et al. “Genetic behavioral screen identifies an orphan anti-opioid system.” Science (2019) 365:1267-1273). Mice were placed in a Plexiglas chamber (16″ tall and 8″ in diameter) on a ceramic plate heated to 52.5° C. and the timer started. Paw licking, paw flicking and jumping were coded as a nociceptive response, upon which the timer was stopped, or up to a maximum of 20 s (for YM-254890 or vehicle) or 50 s (for morphine). Three trials were made with a 3-min intertrial interval. The mean paw withdrawal latency from the three trials was used as the baseline latency. The analgesic effect of drug or vehicle was then determined by a single measurement of paw withdrawal or paw flicking or jumping latencies at respective time intervals. On the test day, mice were first tested for the baseline measurement (t=0, no drug/vehicle given), and the response latency was recorded. The drug (YM-254890 or vehicle or morphine) was given after a few minutes, and the nociceptive response was recorded at 10, 20, 30, 60, 90 and 120 min and up to the time it reached baseline. For the combined administration effect of YM-254890 with morphine, YM-254890 was administered 10 min before the administration of morphine. Time(s) spent on hotplate was graphed as percent maximum possible effect (MPE): MPE (%)=(test latency−baseline latency)/(cut-off time−baseline latency)×100. Nociceptive responses were monitored on alternate days.

(2) Tail Immersion test. Anti-nociception induced by YM-254890 or evaluation of morphine's analgesic effects was determined by tail immersion test. The test was performed as previously described (Wang et al. 2019). Individual mice were transferred to experimentation room and restrained using a well-ventilated 50-ml tube with air holes. In order to minimize handling and to facilitate both the drug delivery and testing, each mouse was comfortably positioned in the plastic restrainer tubes with both fore paws and hind paws extending through holes at the bottom of the restrainer. All animals were habituated to restraint 1 h for 3 days prior to behaviour test. After 3 days of training, the mouse usually voluntarily entered the tube-shaped restrainer during the behavioural testing. No sign of distress was observed in these mice during restraint. Once the animal was immobilized (within 25-30 s), two thirds of the entire tail was dipped in water bath heated to 54° C. Based on our preliminary experiments and reported by others, the tail flick latency was required to be relatively short (2-3 s) for the test to remain valid. The tail flick latency was defined as the time from the onset of thermal heat to tail withdrawal. Three trials were made with a 3-min intertrial interval in between. The mean tail flick latency from the three trials was used as the baseline latency. The analgesic effect of drug or vehicle was then determined by a single measurement of tail flick latencies at respective time intervals. A maximum cut-off was 10 s (for YM-254890 or vehicle) or 20 s (for morphine). The results were then expressed as a percentage of the MPE using the equation described above. On the test day, mice were first tested for the baseline measurement (t=0, no drug/vehicle given), and the response latency was recorded. The drug (YM-254890 or vehicle or morphine) was given after few minutes, and the nociceptive response was recorded at 10, 20, 30, 60, 90 and 120 min and up to the time until baseline was reached. For the effect of combined administration of YM-254890 with morphine, YM-254890 was administered 10 min before the administration of morphine

(3) Open field test. The open-field test was used to measure locomotor activity in animals, where both baseline activity and drug-induced changes can be quantified (Prut L et al. “The open field as a paradigm to measure the effects of drugs on anxiety-like behaviors: A review.” European Journal of Pharmacology (2003) 463:3-33). The distance travelled during the test period was recorded as the index of locomotor activity. Locomotor activity and position within the open field were measured for 2 h. The multiple-unit open-field maze consisted of four activity chambers. Each chamber was made from white high-density and non-porous plastic and measured 50×50×38 cm. Mice were placed individually in the centre of an open-field arena with light bottom to contrast with animal colour, and the test started immediately. Light in the chamber was measured to be 40 1×. The arena was cleaned between each test using alcohol 70% to avoid interference from the smell of the previously tested animal. The exploratory activity was analysed within 2 h. Videos were analysed using an Etho Vision XT16 system (Noldus Information Technology, Wageningen, The Netherlands) considering two previously defined areas: a central and an outer arena.

E. Randomization and blinding. Animals were first assigned a group designation and weighed. Six different cohorts of C57BL/6J animals were used for the four different behavioural testing as reported in systemic (two cohorts for hotplate and tail immersion test), spinal (one cohort), central (one cohort) and open-field test (two cohorts for systemic and spinal administration) paradigms. For each cohort, a total of 12 mice (6 male and 6 female) were divided into two different groups (6 animals: 3 males and 3 females per group). Then each mouse was assigned a temporary random number within that group. Then, the cages were randomized within the experimental group. All recordings were blinded prior to scoring. Experimental results that were not blinded were collected by program software. Program software was calibrated (˜30 min before starting the experiment). No animals were excluded prior to and during studies because all were healthy.

F. Acutely dissociated dorsal root ganglion (DRG) Preparation. Male or female mice between 1 and 2 months of age were used for acute DRG isolations. Animals were killed by CO₂ inhalation, followed by cervical dislocation and postmortem decapitation. Dissociated DRG cultures were prepared as previously described (Perner C et al. “Protocol for dissection and culture of murine dorsal root ganglia neurons to study neuropeptide release.” STAR Protoc (2021) 2:100333). Briefly, DRGs were dissected from 1- to 2-month-old mice in Hank's buffered salt solution (HBSS) and digested with Collagenase A and Dispase II for 25 min at 37° C., centrifuged at 200×g for 5 min, and washed with Dulbecco's modified Eagle's medium (DMEM) supplemented with 10% fetal bovine serum (FBS), glutamate, sodium pyruvate, 1% penicillin and 1% streptomycin. DRGs were then triturated in Neurobasal A medium supplemented with 10% FBS, 1% penicillin, 1% streptomycin, 1% GlutaMAX, 2% B27, 25 ng/L nerve growth factor (NGF) and 2 ng/L glial cell line-derived neurotrophic factor (GDNF). After plating on laminin-coated coverslips, cells were incubated at 37° C. overnight. Experiments were performed the day after dissection.

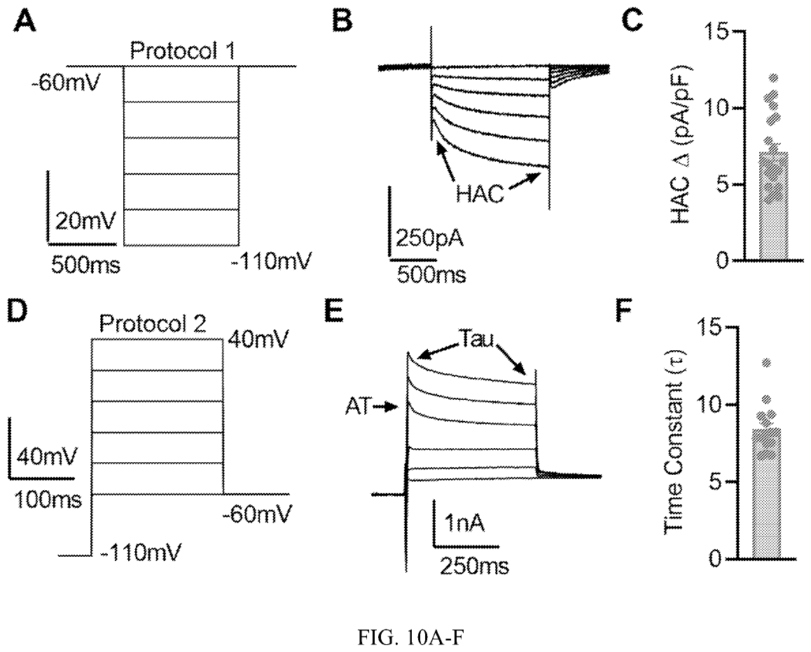

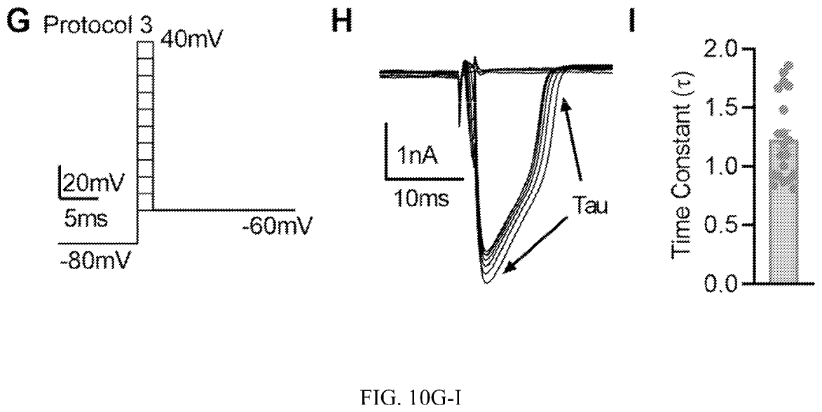

G. Whole cell patch-clamp recordings. Nociceptors were identified by physiological classification protocols (Petruska J C et al. “Subclassified acutely dissociated cells of rat DRG: histochemistry and patterns of capsaicin-, proton-, and ATP-activated currents.” J Neurophysiol (2000) 84:2365-2379) summarized in FIG. 10 and further by responsivity to morphine. Each experimental group consisted of 12-18 DRG recordings from three animals, 1-2 of which were female. Hyperpolarization-activated current (Ih) and kinetics of resulting transient currents were determined by stepping membrane potential from −60 to −110 mV for 500 ms in 10 mV increments (FIG. 10A). Outward currents were identified by first preconditioning membrane potential at −100 mV for 500 ms and then stepping from −60 to 40 mV in 20 mV increments for 200 ms (FIG. 10D). Inward current dynamics were determined by preconditioning at −80 mV and then stepping from −60 to 40 mV in 10 mV increments (FIG. 10G). A 6-s, 0- to 2-nA continuous ramp stimulation protocol was used to determine rheobase. Membrane capacitance (Cm), series resistance (Rs) and input resistance (Ri) were tested with a hyperpolarizing 10 mV pulse of 10 ms duration initially and at a 30 s interval throughout the recording period. Cells were recorded only if initial series resistance was ≤20 MΩ and excluded if Rs varied >20% while recording. Offline analysis identified clustering of a population n=30 with uniform morphine responsivity that was selected for further analysis, with basal parameters #SEM: Cm=27.31±4.72 pF, Ri=492±19.29 MQ, Ih=7.10±2.45 pA·pF−1, IA tau=8.39±0.41 ms and action potential (AP) tau=1.22±0.08 ms. K+ internal was used for all recordings, containing: 6 mM NaCl, 4 mM NaOH, 130 mM K-gluconate, 11 mM ethylene glycol-bis(β-aminoethyl ether)tetraacetic acid (EGTA), 1 mM CaCl₂, 1 mM MgCl₂, 10 mM N-(2-hydroxyethyl)piperazine-N′-(2-ethanesulphonic acid) (HEPES), 2 mM Na₂ATP, and 0.2 mM Na₂GTP 0.2, adjusted to pH 7.3-7.4 with HCl. External artificial CSF contained: 125 mM NaCl, 3 mM KCl, 1.2 mM KH₂PO₄, 1.2 mM MgSO₄, 25 mM NaHCO₃, 2 mM CaCl₂), and 10 mM dextrose 10, adjusted to pH 7.3-7.4 with HCl.

H. Data collection and statistical analysis. Sample sizes appropriate for each type of experiment were estimated on the basis of pilot studies and were calculated on the basis of the equation (Eng J. “Sample size estimation: How many individuals should be studied?” Radiology (2003) 227:309-313): CI95=1.96s/√n, where CI stands for the confidence interval, 1.96 is the corresponding tabulated value for CI95, s is the standard deviation of the mean and n is the sample size. When experiments are novel, it is difficult to perform a priori sample size calculations (Curtis M J et al. “Experimental design and analysis and their reporting: New guidance for publication in BJP.” British Journal of Pharmacology (2015) 172:3461-3471) because the effect size and variance are unknown. Therefore, for animal experiments, estimates of the expected variance and effect size from previous experiments using similar methods were used to estimate appropriate sample sizes a priori through statistical power calculations. Data are presented as mean±SEM. A D'Agostino-Pearson test and Shapiro-Wilk normality tests were applied to evaluate data normality and homogeneity. Parametric statistics for normally distributed variables included unpaired t test and two-way analysis of variance (ANOVA). In addition, group differences using two factors or independent variables were evaluated by two-way ANOVA. Bonferroni's post hoc for multiple comparisons was applied when the main effects of factor were significant in the ANOVA analysis. A non-parametric test (Spearman rank, R) was used to check correlations when one of the variables was not normally distributed. Kruskal-Wallis non-parametric test followed by Dunn's multiple comparisons test was applied for the data that were not normally distributed. For the open-field test, data recorded by Etho Vision software were exported and tabulated in Excel. Thereafter, statistical analysis of these data was carried out using GraphPad Prism software. No animals were excluded from the study, and the data were monitored for statistical outliers. For electrophysiological data, measurements were performed with Clampfit Version 10.5 (Molecular Devices, San Jose, CA, USA). All the statistical analysis was performed using GraphPad Prism Version 9.0.0 for Windows (GraphPad Software, San Diego, CA, USA). Post-hoc tests were run only if F achieved P<0.05 and there was no significant variance inhomogeneity. A P value less than 0.05 (P<0.05) was considered statistically significant.

I. Nomenclature of Targets and Ligands. Key protein targets and ligands in this article can be found in the IUPHAR/BPS Guide to PHARMACOLOGY and are permanently archived in the Concise Guide to PHARMACOLOGY 2019/20.

### Example 2. Effects of Systemic Administration of YM-254890 on Central Nociception and Locomotion

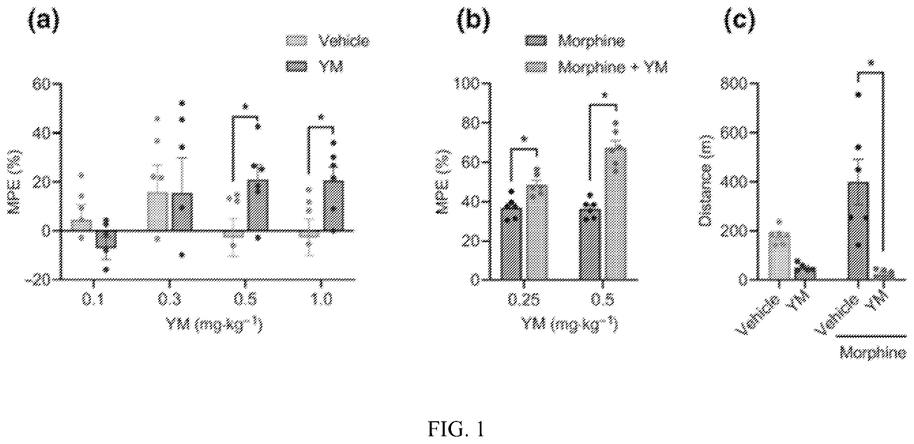

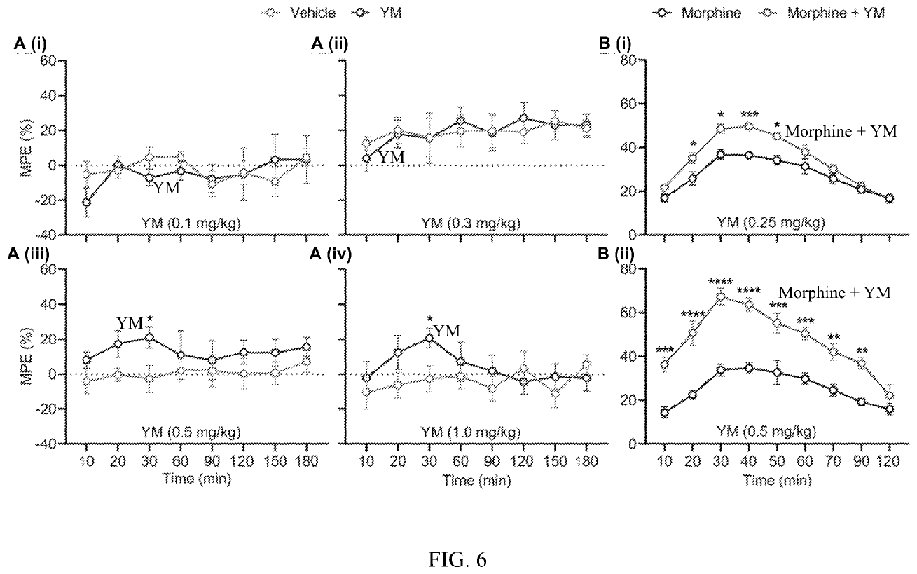

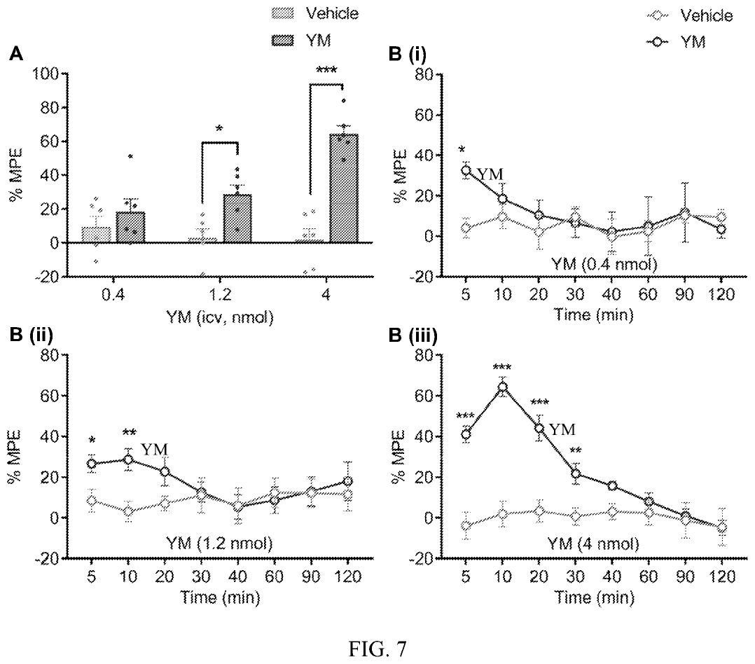

We started by evaluating the effects of YM-254890 on pain responses of mice in the hotplate test by performing the dose-response studies administering the drug systemically via subcutaneous injections. A concentration range of 0.1-1 mg·kg−1 that we explored has been reported to be safe for systemic administration (Meleka M M et al. “Anti-hypertensive mechanisms of cyclic depsipeptide inhibitor ligands for Gα_{q/11} class G proteins.” Pharmacol Res (2019) 141:264-275). We found that treatment with YM-254890 at higher doses (0.5 and 1.0 mg·kg−1) produced increases in paw withdrawal latencies reaching maximal analgesic response at 30 min (unpaired t test, P<0.05) after injection (FIGS. 1A and 6A). No significant effect of YM-254890 at lower doses (0.1 and 0.3 mg·kg−1) (FIGS. 1A and 6A) was observed. To confirm that YM-254890 directly affected the central nervous system to produce analgesic effects, the drug was delivered directly into the brain. A dose-dependent increase in both the extent and duration of analgesia upon YM-254890 administration (FIG. 7) was observed.

The effect of YM-254890 treatment on opioid-induced analgesia was evaluated. Administration of subthreshold dose of YM-254890 (0.25 mg·kg−1) significantly potentiated anti-nociceptive effects in the hotplate test (FIGS. 1B and 6B). Increasing the dose (YM-254890 0.5 mg·kg−1) led to further enhancement of opioid analgesia (FIGS. 1B and 6B). Interestingly, although YM-254890 treatment increased the maximal degree of the effect, it had no effect on either the onset timing or duration of the opioid analgesia with all the animals reaching maximal latencies in about 30 min and recovering to the baseline nociceptive thresholds in about 3 h.

To evaluate effects of YM-254890 treatment on locomotion behaviour, mice were tested in the open-field test. YM-254890 was injected at the doses that produced the most significant analgesic effects both alone and in combination with morphine. Analysis of the results indicated that although morphine alone produced well-known locomotor-sensitizing effect, co-treatment with the YM-254890 completely suppressed these effects and resulted in substantial decrease in locomotion comparable with the levels seen with the YM-254890 treatment alone (FIG. 1C).

### Example 3. Effects of Systemic Gα_{q/11} Inhibition on Spinal Analgesia

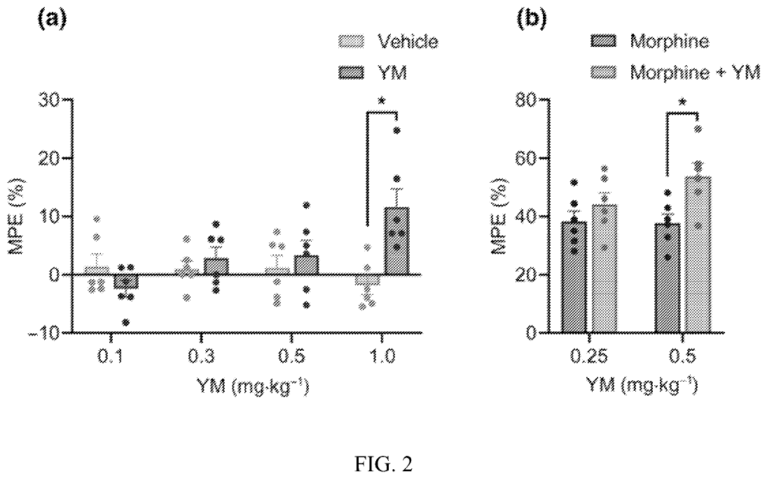

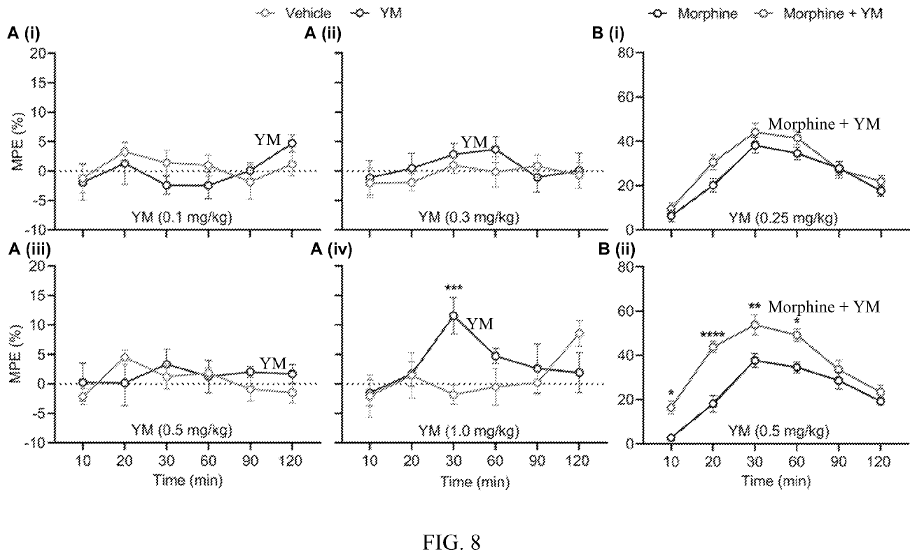

Because changes in locomotor activity can confound the interpretation of the hotplate test, which relies on complex body movements, we next used the tail flick test, which relies on spinal reflexes to assess nociception in mice. We found that YM-254890 induced a significant anti-nociceptive response at the highest dose of 1 mg·kg−1 (FIGS. 2A and 8A). However, no significant difference in nociceptive response was observed with lower doses (FIGS. 2A and 8A).

Next, we evaluated the effect of YM-254890 treatment at subthreshold doses of morphine analgesia using the same tail immersion test (FIG. 2B). We observed significant enhancement of the anti-nociceptive effects of morphine at 0.5-mg·kg−1 dose of YM-254890 (FIGS. 2B and 8B). Reducing the dose to 0.25 mg kg-1 eliminated this effect (FIGS. 2B and 8B). In summary, these results indicate that when administered systemically, YM-254890 has analgesic properties.

### Example 4. Analgesic Properties of Intrathecal YM-254890 Treatment and its Synergy with Opioids

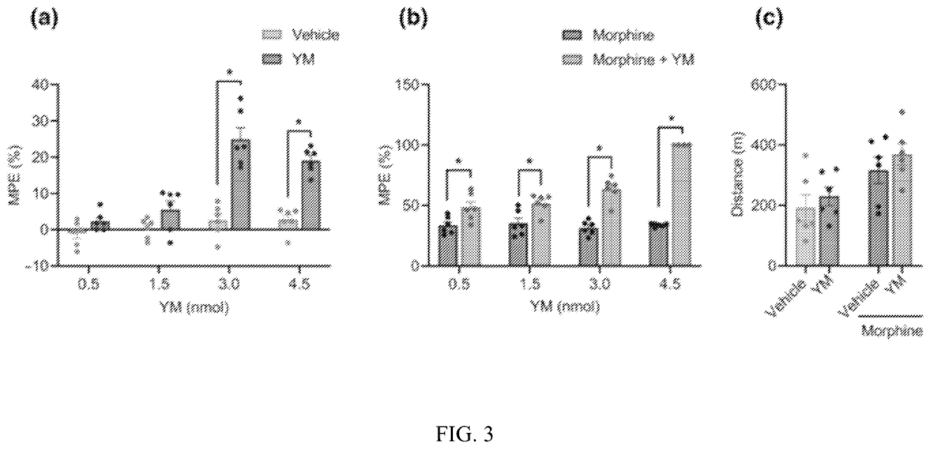

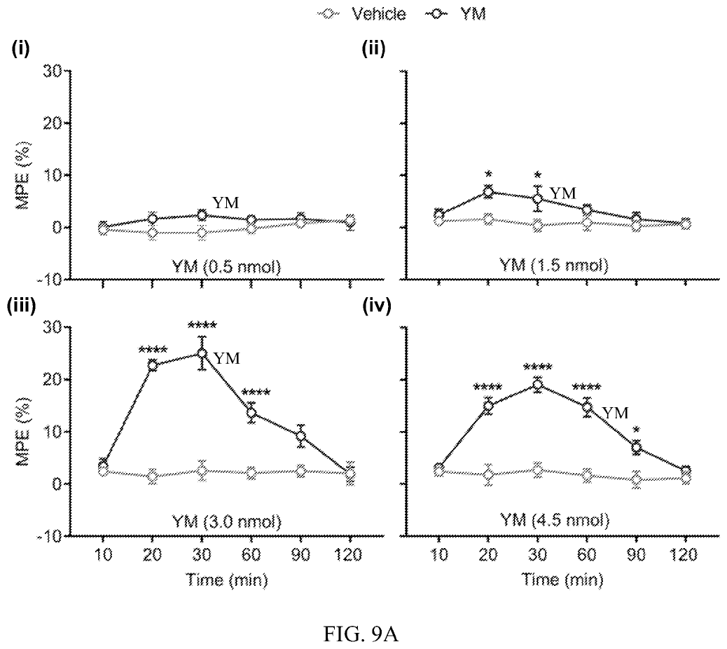

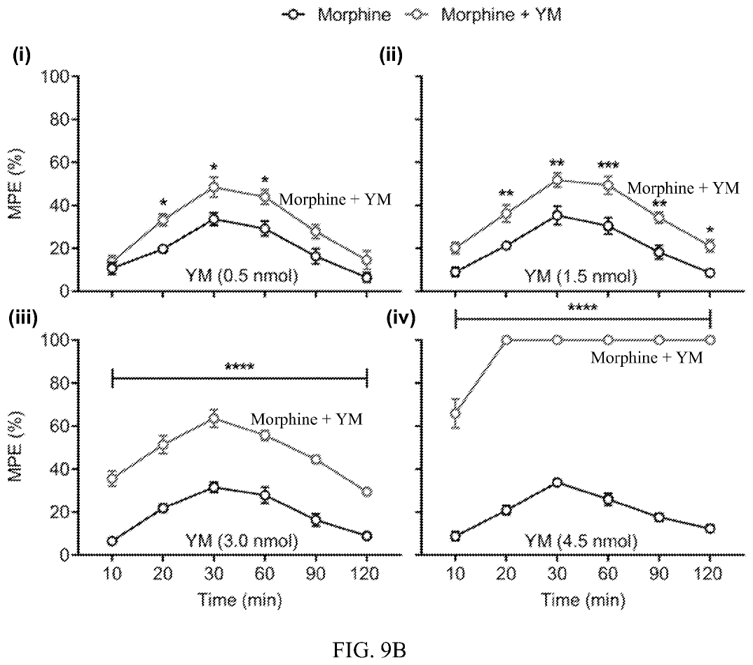

We explored spinal intrathecal delivery, a route routinely used in clinical practice for mitigating unwanted effects especially in the context of opioid treatment (Fairbanks CA “Spinal delivery of analgesics in experimental models of pain and analgesia.” Adv Drug Deliv Rev (2003) 55:1007-1041). When injected intrathecally, YM-254890 showed significant anti-nociceptive properties in the tail immersion test at doses above 1.5 nmol per injection (FIGS. 3A and 9A). The magnitude of the effect did not change upon increasing the dose to 4.5 nmol suggesting the ceiling effect. The duration of the effect did not differ between the two maximally effective doses (3.0 and 4.5 nmol) with the animals returning to baseline nociception in about 2 h (FIGS. 3A and 9A).

The effects of intrathecal YM-254890 administration on systemic morphine analgesia were tested in the tail immersion test. The lowest subthreshold dose of 0.5 nmol was observed to significantly enhance the anti-nociceptive effects of morphine (FIGS. 3B and 9B). This effect was increased dose dependently until maximizing at the cut-off value for the test (FIG. 9B). At each of the doses, both the maximal extent and duration of the analgesic effects of morphine were increased. Because inhibition of motor activity was a confound of the YM-254890-induced analgesia upon systemic administration, we further monitored its effects on animal locomotion following intrathecal delivery. As expected from local delivery method, we found no significant changes in motor activity induced by YM-254890 when administered either alone or in combination with morphine (FIG. 3C). Together, these results indicate effectiveness of YM-254890 as an analgesic at the level of the spinal cord and its synergy with opioid-induced analgesia.

### Example 5. Gα_{q/11} Inhibition Suppresses Activity of DRG Nociceptors and Augments their Responsiveness to Opioid Inhibition

To obtain insights into the mechanisms by which inhibition of Gq/11 produces analgesic effects and enhances morphine action, we examined the impact of YM-254890 on electrophysiological properties of DRG nociceptors in the peripheral nervous system. These neurons play a crucial role in nociception and express the direct target of opioid analgesics—the μ receptor.

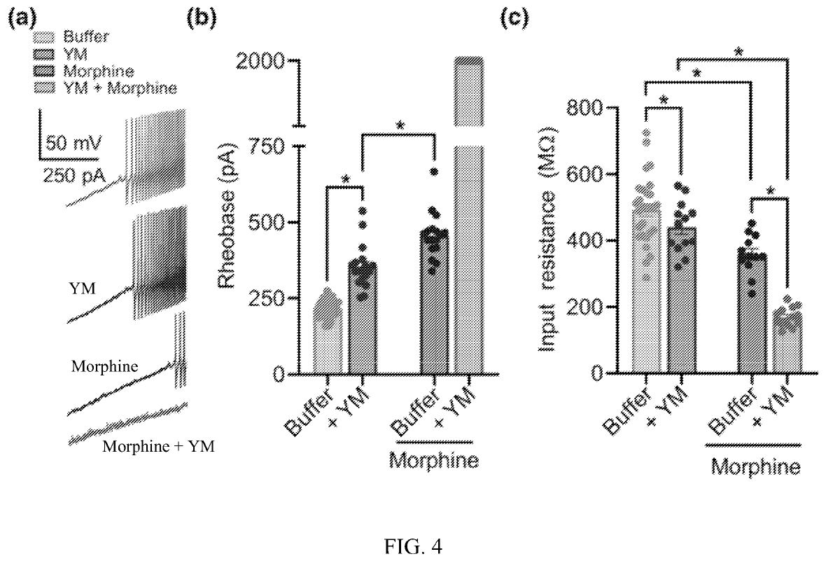

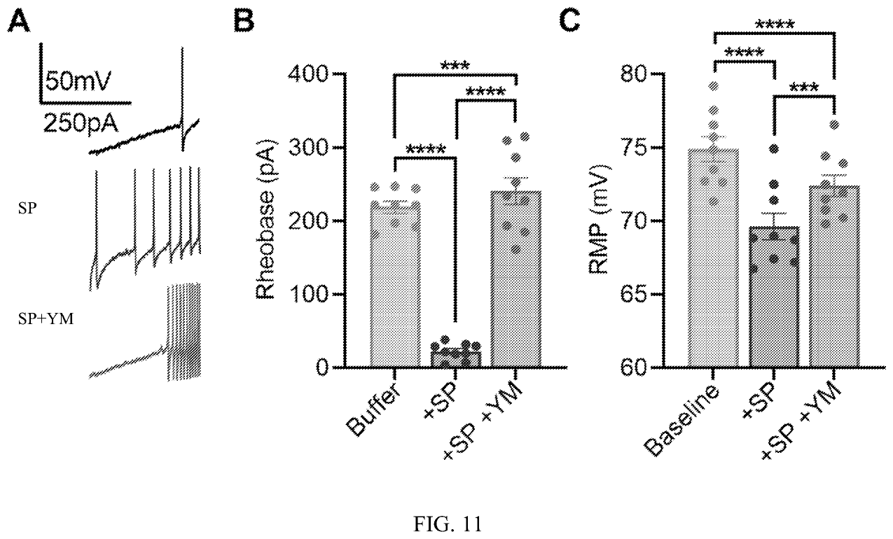

DRG nociceptors were identified by multiple electrophysiological characteristics (FIG. 10) including presence of a hyperpolarization-activated current, A-type current inactivation rate, inward current dynamics, and responsiveness to morphine. The effectiveness of YM-254890 as a Gq/11 antagonist in this population was verified by its ability to inhibit the effects of substance P (neurotransmitter 11-mer peptide), which mediates its effects via canonical Gq/11-coupled pathway (FIG. 11). Consistent with prior observations, application of morphine substantially decreased excitability of these DRG neurons as evidenced by a significant increase in rheobase when using ramp stimulation protocol (FIG. 4A-B). Treatment with YM-254890 alone also caused significant decrease in the excitability of DRG nociceptors (FIG. 4A-B). The magnitude of this effect was smaller as compared with morphine, consistent with the lower analgesic efficacy of YM-254890 relative to morphine observed in behavioural studies. A similar interaction was observed with resting input resistance (FIG. 4C).

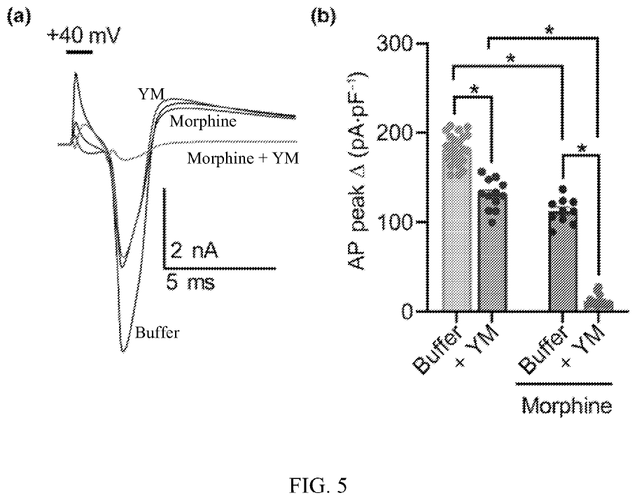

To better understand these effects, AP dynamics with a voltage-step protocol were examined (FIG. 10G-I). Application of either YM-254890 or morphine moderately reduced AP amplitude to approximately similar extents (FIG. 4C-D). However, co-application of YM-254890 and morphine largely prevented evoked AP responses (FIG. 5A-B). This synergistic interaction was also apparent in the modulation of rapidly inactivating A-type potassium currents (IA) tested with voltage-step protocol 2 (FIG. 10D-F). Both YM-254890 and morphine significantly inhibited IA, whereas their coadministration nearly eliminated IA. These physiological effects demonstrate that YM-254890 and morphine interact synergistically to reduce nociceptor excitability as a mechanism for producing analgesic effects.

1. A method for treating pain in a subject comprising administering an effective amount of a Gα_{q/11} inhibitor to the subject.

2. The method of claim 1, wherein administering a Gα_{q/11} inhibitor provides or enhances an analgesic effect mediated by a μ-opioid receptor.

3. The method of claim 1, wherein the subject is a human.

4. The method of claim 1, wherein Gα_{q/11} inhibitor comprises YM-254890, FR-900359, GQ262, or GQ127.

5. The method of claim 1, wherein Gα_{q/11} inhibitor comprises a Gα_{q/11} RNA interference (RNAi) polynucleotide, a Gα_{q/11} antisense oligonucleotide (ASO), or a Gα_{q/11} CRISPR system.

6. (canceled)

7. The method of claim 1, wherein the Gα_{q/11} inhibitor is administered by systemic administration, intravenous administration, subcutaneous administration, local administration, epidural administration, or intrathecal administration.

8. The method of claim 1, wherein the method further comprises administering an opioid drug to the subject.

9. The method of claim 8, wherein the opioid drub comprises oxycodone, hydrocodone, morphine, codeine, dihydrocodeine, fentanyl, pethidine, hydromorphone, buprenorphine, or methadone

10. A method of increasing efficacy of an opioid therapy in a subject comprising administering an effective amount of a Gα_{q/11} inhibitor to the subject.

11. The method of claim 10, wherein the subject is administered an opioid drug for pain relief.

12. The method of claim 11, wherein the Gα_{q/11} inhibitor is administered to the subject prior to an initial dose of the opioid drug, at the same time as an initial dose of the opioid drug, at the same time as one or more subsequent doses of the opioid drug, or after an initial dose of the opioid drug.

13. The method of claim 11, wherein the opioid drug is oxycodone, hydrocodone, morphine, codeine, dihydrocodeine, fentanyl, pethidine, hydromorphone, buprenorphine, or methadone.

14. The method of claim 10, wherein increasing efficacy of an opioid therapy comprises: enhancing maximal response to the opioid, increasing response to a given dose of opioid, enhancing the analgesic effect of low dose of the opiate, decreasing the amount of the opioid required to provide an effective analgesic response, increasing duration of analgesic response to the opioid, and/or decreasing tolerance of the opioid.

15. The method of claim 10, wherein increasing efficacy of an opioid therapy comprises: increasing safety of an opioid, and/or decreasing risk of addiction or dependence on the opioid drub.

16. The method of claim 10, wherein the subject is a human.

17. The method of claim 10, wherein Gα_{q/11} inhibitor comprises YM-254890, FR-900359, GQ262, or GQ127.

18. The method of claim 10, wherein Gα_{q/11} inhibitor comprises a Gα_{q/11} RNA interference (RNAi) polynucleotide, a Gα_{q/11} antisense oligonucleotide (ASO), a Gα_{q/11} CRISPR system, or an anti-Gα_{q/11} antibody or Gα_{q/11}-binding fragment thereof

19. The method of claim 10, wherein the Gα_{q/11} inhibitor is administered by systemic administration, intravenous administration, subcutaneous administration, local administration, epidural administration, or intrathecal administration.

20. The method of claim 10, wherein the Gα_{q/11} inhibitor comprises YM-254890, wherein the YM-254890 is delivered by epidural administration or intrathecal administration.

21. A method for suppressing excitability of DRG nociceptors in a subject comprising, administering a Gα_{q/11} inhibitor or a Gα_{q/11} inhibitor and an opioid analgesic to the subject.
